SEQUENCE LISTING (4 sequences)

SEQ ID NO: 1 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS

SEQ ID NO: 3 - protein, 238 aa, Homo sapiens
IVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 4 - protein, 238 aa, Homo sapiens
IVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS

(19) United States
(12) Patent Application Publication
(10) Pub. No.: US 2025/0319167 A1
(43) Pub. Date: Oct. 16, 2025

(54) INTRAVENOUS COMPOSITIONS OF TISSUE KALLIKREIN-1 AND RELATED METHODS

(71) Applicant: DiaMedica Therapeutics, Inc., Minneapolis, MN (US)

(72) Inventors: Ambarish SHAH, Minneapolis, MN (US); Nick PAULSON, St. Paul, MN (US)

(21) Appl. No.: 19/178,631

(22) Filed: Apr. 14, 2025

Related U.S. Application Data
(60) Provisional application No. 63/634,223, filed on Apr. 15, 2024.

(51) Int. Cl. A61K 38/48; A61K 9/00; A61K 47/26

(52) U.S. Cl. CPC A61K 38/4853; A61K 9/0019; A61K 47/26; C12Y 304/21035

(57) ABSTRACT

Provided are pharmaceutical compositions, kits, and methods for the clinical use of tissue kallikrein-1 (KLK1) polypeptides in polyolefin-containing intravenous (IV) systems, including polyolefin IV bags.

### CROSS-REFERENCE TO RELATED APPLICATIONS

This application claims the benefit under 35 U.S.C. § 119 (e) of U.S. Provisional Application No. 63/634,223, filed Apr. 15, 2024, which is incorporated by reference in its entirety.

### STATEMENT REGARDING THE SEQUENCE LISTING

The Sequence Listing XML associated with this application is provided in XML file format and is hereby incorporated by reference into the specification. The name of the XML file containing the Sequence Listing XML is DIAM_043_01US_ST26.xml. The XML file is about 5,494 bytes, was created on Apr. 3, 2025, and is being submitted electronically via USPTO Patent Center.

### BACKGROUND

### Technical Field

The present disclosure relates to pharmaceutical compositions, kits, and methods for the clinical use of tissue kallikrein-1 (KLK1) polypeptides in polyolefin-containing intravenous (IV) systems, including polyolefin IV bags.

### Description of the Related Art

Protein therapy with tissue kallikrein-1 (KLK1) has the potential to treat multiple diseases. For instance, KLK1 proteins such as the investigational drug DM199 are intended to restore normal levels of the naturally-occurring protein KLK1, which acts on interrelated mechanisms in the blood vessels and kidneys to improve circulation and overall function (see, for example, PCT/US2018/021749; and PCT/CA2013/050425).

Polyolefin intravenous (IV) bags are in common use as a safer, more environmentally friendly, and autoclavable alternative to polyvinyl chloride (PVC) IV bags, among others. However, investigational clinical studies with KLK1 have observed that the proteins persistently adsorb to the interior surface of polyolefin IV bags, resulting in an inconsistent and overall lower dosage of KLK1 in the administered IV infusion than otherwise planned. Thus, there is a need for improved KLK1 protein-based compositions and methods in the polyolefin-related IV context.

### BRIEF SUMMARY

Embodiments of the present disclosure relate to pharmaceutical compositions, comprising:
(i) one or more tissue kallikrein (KLK1) polypeptides; and
(ii) a polysorbate surfactant (PS) optionally selected from PS20 and PS80.

In certain embodiments, (i) is at a concentration of about 10 μg/mL to about 2000 μg/mL, optionally at a concentration of about 100 μg/mL. In some embodiments, (ii) is at a concentration of about 0.01% to about 2.0%. In certain embodiments, (ii) is at a concentration of about 0.43% to about 0.87% (optionally for use with 50 mL IV bag). In certain embodiments, (ii) is at a concentration of about 0.43% to about 0.87% to about 1.74% (optionally for use with 100 mL IV bag). In certain embodiments, (ii) is PS20. In some embodiments, (ii) is PS80.

Particular pharmaceutical compositions are in a vial at a total volume of about 0.1 mL to about 5 mL, optionally about 0.15 mL to about 0.5 mL. In specific embodiments, (i) is at concentration of about 100 μg/mL, (ii) is at a concentration of about 0.43% to about 0.87% (for instance, for use with 50 mL IV bag), and the composition is in a vial at a total volume of about 0.15 mL to about 0.5 mL; or (i) is at concentration of about 100 μg/mL, (ii) is at a concentration of about 0.86% to about 1.74% (for example, for use with 100 mL IV bag), and the composition is in a vial at a total volume of about 0.15 mL to about 0.5 mL. In certain embodiments, (i) is DM199.

Some pharmaceutical compositions described herein are for use in a method of diluting the pharmaceutical composition into an IV solution in a polyolefin IV bag, including wherein the IV solution/polyolefin IV bag comprising the diluted pharmaceutical composition is for use in treating a disease or condition in a subject in need thereof, for example, at a total KLK1 polypeptide intravenous dosage of about 0.1 μg/kg to about 0.25 μg/kg to about 0.5 μg/kg to about 5 μg/kg or to about 10.0 μg/kg. Certain pharmaceutical compositions described herein are for use in the preparation of a medicament for treating a disease or condition in a subject in need thereof, including wherein preparation of the medicament comprises diluting the pharmaceutical composition into an IV solution in a polyolefin IV bag, for example, at a total KLK1 polypeptide intravenous dosage of about 0.1 μg/kg to about 0.25 μg/kg to about 0.5 μg/kg to about 5 μg/kg or to about 10.0 μg/kg.

Also included are methods of preparing a polyolefin intravenous (IV) bag for intravenous administration to a subject in need thereof, wherein the polyolefin IV bag comprises an IV solution, comprising:
(a) adding the pharmaceutical composition of any one of claims 1-10 to the IV solution in the polyolefin IV bag; and
(b) inverting or agitating the polyolefin IV bag for a time sufficient to disperse the pharmaceutical composition into a final IV solution.

In certain embodiments, the IV solution is a normal saline (0.9% sodium chloride), a half-strength normal saline (0.45% sodium chloride), a dextrose solution (optionally 5% in water), or a Lactated Ringer's (LR) solution. In some embodiments, the polyolefin IV bag with IV solution has a total volume of about 50-1000 ml, optionally about 50 ml, about 100 ml, about 250 ml, about 500 ml, or about 1000 ml. In certain embodiments, the one or more KLK1 polypeptides of (i), optionally DM199, are in the final IV solution at a concentration of about 0.03 μg/mL to about 15.0 μg/mL, optionally about 0.15 μg/mL to about 1.0 μg/mL. In certain embodiments, the polysorbate surfactant of (ii) is in the final IV solution at a concentration of about 0.0001% to about 0.10%, optionally about 0.001% to about 0.002%. In certain embodiments, the polysorbate surfactant of (ii) is PS20. In certain embodiments, the polysorbate surfactant of (ii) is PS80.

In specific embodiments, the pharmaceutical composition of step (a) comprises the one or more KLK1 polypeptides of (i), optionally the DM199, at concentration of about 100 μg/mL, and the PS surfactant of (ii) at a concentration of about 0.43% to about 0.87%, and wherein the polyolefin IV bag with the final IV solution of step (b) has a total volume of about 50 mL, the one or more KLK1 polypeptides, optionally DM199, are in the final IV solution at a concentration of about 0.15 μg/mL to about 1.0 μg/mL, and the PS surfactant of (ii) is in the final IV solution at a concentration of about 0.001% to about 0.002%; or the pharmaceutical composition of step (a) comprises the one or more KLK1 polypeptides of (i), optionally DM199, at concentration of about 100 μg/mL, and the PS surfactant of (ii) at a concentration of about 0.86% to about 1.74%, and wherein the polyolefin IV bag with the final IV solution of step (b) has a total volume of about 100 mL, wherein the one or more KLK1 polypeptides of (i), optionally the DM199, are in the final IV solution at a concentration of about 0.15 μg/mL to about 1.0 μg/mL, and the PS surfactant is in the final IV solution at a concentration of about 0.001% to about 0.002%.

Some embodiments comprise the step (c) intravenously administering the final IV solution to the subject in need thereof via the polyolefin IV bag, for instance, at a total KLK1 polypeptide intravenous dosage of about 0.1 μg/kg to about 0.25 μg/kg to about 0.5 μg/kg to about 5 μg/kg or to about 10.0 μg/kg. Also included are methods of treating a disease or condition in a subject in need thereof, comprising administering a final IV solution to the subject in need thereof via the polyolefin IV bag as prepared according to the description herein, for example, at a total KLK1 polypeptide intravenous dosage of about 0.1 μg/kg to about 0.25 μg/kg to about 0.5 μg/kg to about 5 μg/kg or to about 10.0 μg/kg.

Also included are patient care kits, comprising:
(i) a first vial comprising a pharmaceutical composition, wherein the pharmaceutical composition comprises one or more tissue kallikrein (KLK1) polypeptides; and (ii) a second vial comprising a polysorbate surfactant optionally selected from PS20 and PS80.

In certain embodiments, the one or more KLK1 polypeptides in the pharmaceutical composition of (i) are at a concentration of about 10 μg/mL to about 500 μg/mL, optionally at a concentration of about 100 μg/mL. In some embodiments, the polysorbate surfactant of (ii) is at a concentration of about 0.005% to about 5.0%, optionally about 0.1% to about 0.2% to about 0.4% to about 0.8%. In certain embodiments, the polysorbate surfactant of (ii) is at a concentration of about 0.1% (optionally for use with 50 mL IV bag). In certain embodiments, the polysorbate surfactant of (ii) is at a concentration of about 0.2% (optionally for use with 100 mL IV bag). In some embodiments, the polysorbate surfactant of (ii) is PS20. In particular embodiments, the polysorbate surfactant of (ii) is PS80.

In certain embodiments, the first vial is at a total volume of about 0.1 mL to about 5 mL, optionally about 0.15 mL to about 0.5 mL, and the second vial is at a total volume of about 0.25 mL to about 0.5 mL to about 1.0 ml to about 2.0 mL, optionally about 1 mL. In certain embodiments, the first vial is at a total volume of about 0.15 mL to about 0.5 mL and comprises the pharmaceutical composition of one or more KLK1 polypeptides, optionally DM199, at a concentration of about 100 μg/mL, and the second vial is at a total volume of about 0.25 mL to about 2.0 mL and comprises the polysorbate surfactant at a concentration of about 0.1% to about 0.8%, optionally wherein the second vial is about 2 mL total volume/about 0.1% surfactant, about 1 mL total volume/about 0.2% surfactant, about 0.5 mL total volume/about 0.4% surfactant, or about 0.25 mL total volume/about 0.8% surfactant. In certain embodiments, the pharmaceutical composition of (i) comprises DM199.

Some patient care kits described herein are for use in a method of diluting the first vial and the second vial into an IV solution in a polyolefin IV bag, optionally wherein the IV solution/polyolefin IV bag comprising the diluted first and second vials is for use in treating a disease or condition in a subject in need thereof. Also included is the use of a patient care kit described herein in the preparation of a medicament for treating a disease or condition in a subject in need thereof, wherein preparation of the medicament comprises diluting the first vial and the second vial into an IV solution in a polyolefin IV bag.

Also included are methods of preparing a polyolefin intravenous (IV) bag for intravenous administration to a subject in need thereof, wherein the polyolefin IV bag comprises an IV solution, comprising:
(a) adding a polysorbate surfactant to the IV solution in the IV bag, wherein the polysorbate surfactant is selected from PS20 and PS80;
(b) separately adding a pharmaceutical composition comprising one or more tissue kallikrein (KLK1) polypeptides to the IV solution in the polyolefin IV bag; and
(c) inverting or agitating the IV bag for a time sufficient to disperse the pharmaceutical composition and the polysorbate surfactant into a final IV solution.

In certain embodiments, the IV solution is a normal saline (0.9% sodium chloride), a half-strength normal saline (0.45% sodium chloride), a dextrose solution (optionally 5% in water), or a Lactated Ringer's (LR) solution. In some embodiments, the polyolefin IV bag with IV solution has a total volume of about 50-1000 ml, optionally about 50 ml, about 100 ml, about 250 ml, about 500 ml, or about 1000 ml. In certain embodiments, the polysorbate surfactant in (a) is added to the final IV solution of (c) at a concentration of about 0.0001% to about 0.10%, optionally about 0.001% to about 0.002%. In particular embodiments, the polysorbate surfactant of (ii) is PS20. In certain embodiments, the polysorbate surfactant of (ii) is PS80.

In certain embodiments, the one or more KLK1 polypeptides in (b), optionally DM199, are added to the final IV solution of (c) at a concentration of about 0.03 μg/mL to about 15.0 μg/mL, optionally about 0.15 μg/mL to about 1.0 μg/mL. In certain embodiments, the polyolefin IV bag with the final IV solution of (c) has a total volume about 50 ml or 100 mL, the polysorbate surfactant added from (a) is in the final IV solution at a concentration of about 0.001% to about 0.002%, and the one or more KLK1 polypeptides added from (b), optionally DM199, are in the final IV solution at a concentration of about 0.15 μg/mL to about 1.0 μg/mL. Certain methods are performed using a patient care kit described herein.

Some embodiments comprise the step (d) intravenously administering the final IV solution to the subject in need thereof via the polyolefin IV bag, for instance, at a total KLK1 polypeptide intravenous dosage of about 0.1 μg/kg to about 0.25 μg/kg to about 0.5 μg/kg to about 5 μg/kg or to about 10.0 μg/kg.

Also included are methods of treating a disease or condition in a subject in need thereof, comprising administering a final IV solution to the subject in need thereof via the polyolefin IV bag as prepared according to the description herein, for instance, at a total KLK1 polypeptide intravenous dosage of about 0.1 μg/kg to about 0.25 μg/kg to about 0.5 μg/kg to about 5 μg/kg or to about 10.0 μg/kg.

Particular embodiments include a polyolefin intravenous (IV) bag, comprising a final IV solution of:
(i) one or more tissue kallikrein (KLK1) polypeptides; and
(ii) a polysorbate surfactant selected from PS20 and PS80.

In certain embodiments, the IV solution is a normal saline (0.9% sodium chloride), a half-strength normal saline (0.45% sodium chloride), a dextrose solution (optionally 5% in water), or a Lactated Ringer's (LR) solution. In certain embodiments, the IV bag with solution has a total volume of about 50-1000 ml, optionally about 50 ml, about 100 ml, about 250 ml, about 500 ml, or about 1000 ml. In certain embodiments, the one or more KLK1 polypeptides of (i), optionally DM199, are in the final IV solution at a concentration of about 0.03 μg/mL to about 15.0 μg/mL, optionally about 0.15 μg/mL to about 1.0 μg/mL. In certain embodiments, the polysorbate surfactant of (ii) is in the final IV solution at a concentration of about 0.0001% to about 0.10%, optionally about 0.001% to about 0.002%. In certain embodiments, the polysorbate surfactant of (ii) is PS20. In certain embodiments, the polysorbate surfactant of (ii) is PS80.

In certain embodiments, the final IV solution has a total volume about 50 ml or 100 mL, the one or more KLK1 polypeptides from (i), optionally DM199, are in the final IV solution at a concentration of about 0.15 μg/mL to about 1.0 μg/mL, and the polysorbate surfactant from (ii) is in the final IV solution at a concentration of about 0.001% to about 0.002%. In certain embodiments, (i) is DM199.

Some polyolefin IV bags described herein are for use in treating a disease or condition in a subject in need thereof, for instance, at a total KLK1 polypeptide intravenous dosage of about 0.1 μg/kg to about 0.25 μg/kg to about 0.5 μg/kg to about 5 μg/kg or to about 10.0 μg/kg. Also included is the use of a polyolefin IV bag described herein in the preparation of a medicament for treating a disease or condition in a subject in need thereof, for example, at a total KLK1 polypeptide intravenous dosage of about 0.1 μg/kg to about 0.25 μg/kg to about 0.5 μg/kg to about 5 μg/kg or to about 10.0 μg/kg. Also included are methods of treating a disease or condition in a subject in need thereof, comprising administering a final IV solution to the subject in need thereof via a polyolefin IV bag described herein, for instance, at a total KLK1 polypeptide intravenous dosage of about 0.1 μg/kg to about 0.25 μg/kg to about 0.5 μg/kg to about 5 μg/kg or to about 10.0 μg/kg.

In certain embodiments, the one or more KLK1 polypeptides comprise a first KLK1 polypeptide and a second KLK1 polypeptide, wherein the first KLK1 polypeptide has three glycans attached at three different positions per polypeptide and the second KLK1 polypeptide has two glycans attached at two different positions per polypeptide, and wherein the first KLK1 polypeptide and the second KLK1 polypeptides are present at a ratio of about 45:55 to about 55:45. In certain embodiments, the one or more of the glycans are N-linked glycans. In certain embodiments, the one or more of the glycans are attached at amino acid residues 78, 84, or 141 of KLK1 as defined by SEQ ID NO: 3 or 4. In some embodiments, the three glycans of the first KLK1 polypeptide are N-linked glycans at residues 78, 84, and 141. In particular embodiments, the two glycans of the second KLK1 polypeptide are N-linked glycans at residues 78 and 84 but not 141. In certain embodiments, the first KLK1 polypeptide and the second KLK1 polypeptides are present at a ratio of about 50:50.

In certain embodiments, the one or more KLK1 polypeptide(s) are recombinant KLK polypeptides, mature KLK1 polypeptides, human KLK1 (hKLK1) polypeptides, or any combination thereof. In some embodiments, the hKLK1 polypeptide(s) comprise, consist, or consist essentially of amino acid residues 78-141 of SEQ ID NO: 1 or amino acids residues 78-141 SEQ ID NO: 2, or an active fragment or variant thereof having at least 90, 95, 96, 97, 98, or 99% sequence identity to amino acid residues 78-141 of SEQ ID NO: 1 or amino acids residues 78-141 SEQ ID NO: 2 In particular embodiments, the hKLK1 polypeptide(s) comprise, consist, or consist essentially of amino acid residues 25-262 of SEQ ID NO: 1 or amino acid residues 25-262 of SEQ ID NO: 2, or an active fragment or variant thereof having at least 90, 95, 96, 97, 98, or 99% sequence identity to amino acid residues 25-262 of SEQ ID NO: 1 or amino acid residues 25-262 of SEQ ID NO: 2. In certain embodiments, the KLK1 polypeptide(s) comprise E145 and/or A188. In certain embodiments, the KLK1 polypeptide(s) comprise Q145 and/or V188. In some embodiments, the KLK1 polypeptide(s) comprise, consist, or consist essentially of SEQ ID NO: 3 or 4, or an active fragment or variant thereof having at least 90, 95, 96, 97, 98, or 99% sequence to SEQ ID NO: 3 or 4. In specific embodiments, the KLK1 polypeptide(s) comprise, consist, or consist essentially of SEQ ID NO: 4.

In some embodiments, the subject in need thereof has an ischemic condition, optionally selected from one or more of brain ischemia (ischemic stroke), cardiac ischemia (myocardial ischemia), ischemic colitis, limb ischemia, and cutaneous ischemia. In certain embodiments, the subject in need thereof has a chronic kidney disease (CKD), acute kidney injury, or a cardio-renal disease. In certain embodiments, the subject in need thereof has a hemorrhagic condition, optionally hemorrhagic stroke, including intracerebral (within the brain) hemorrhagic stroke and subarachnoid hemorrhagic stroke. In certain embodiments, the subject in need thereof has an inflammatory disease or condition. In certain embodiments, the subject in need thereof has a disease or condition selected from (essential) hypertension, dementia optionally vascular dementia, and mild cognitive impairment (MCI). In certain embodiments, the subject in need thereof has a pregnancy disorder, optionally fetal growth restriction (FGR), preeclampsia, eclampsia, post-partum preeclampsia, chronic hypertension, chronic hypertension superimposed with preeclampsia, and gestational hypertension.

### BRIEF DESCRIPTION OF THE DRAWINGS

FIG. 1 shows a chromatographic overlay of 0%, 0.003%, 0.020%, and 0.100% PS20 in 1.0 μg/ml Sample Solutions (T=0).

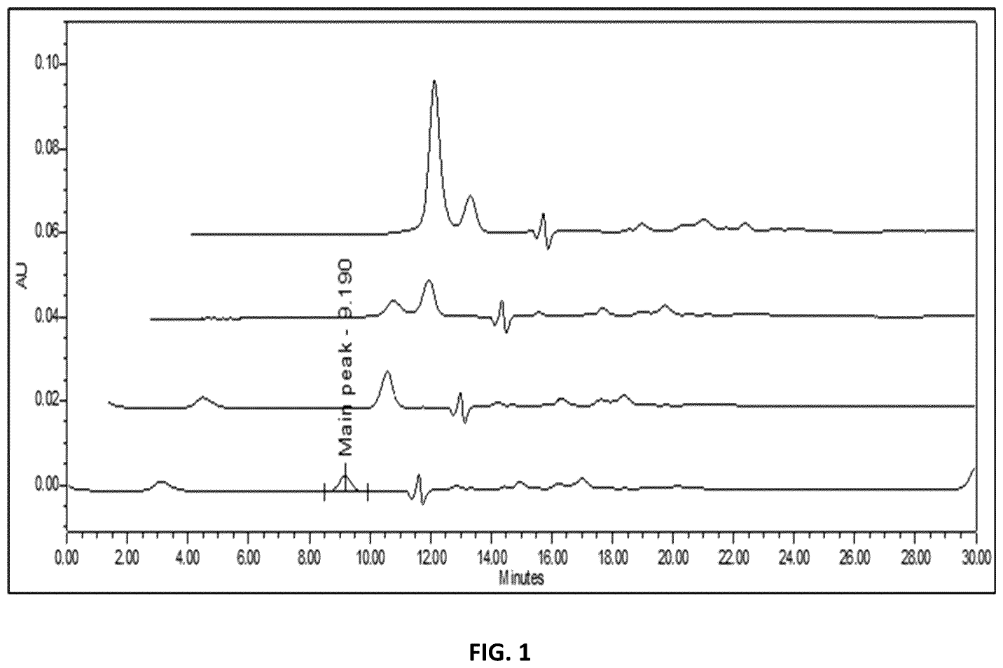

FIG. 2 shows a chromatographic overlay of 0%, 0.003%, 0.020%, and 0.100% PS20 in 1.0 μg/ml Sample Solutions (T=8).

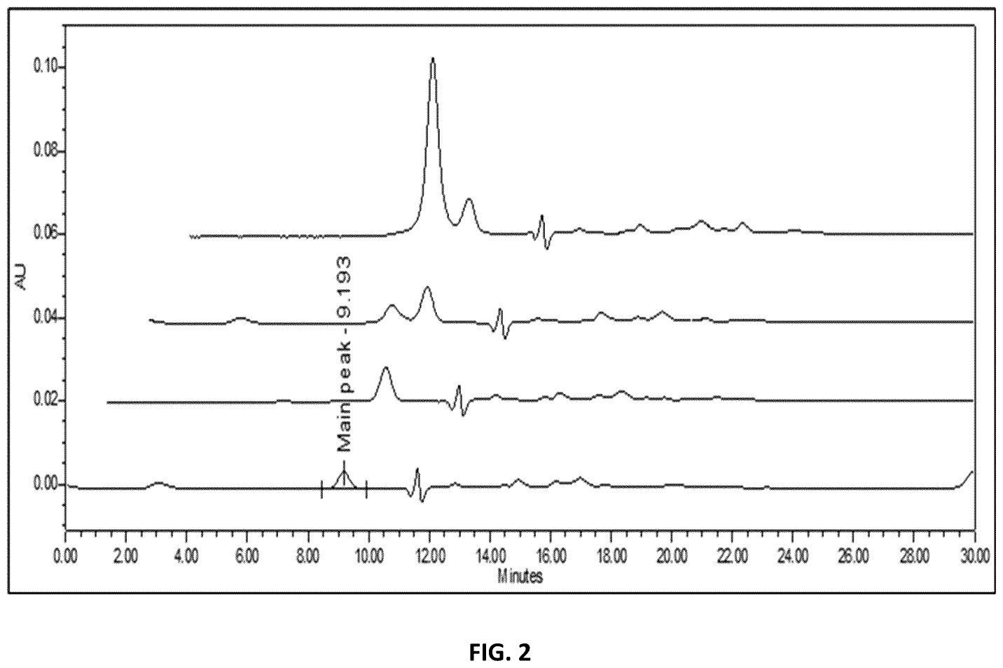

FIG. 3 shows a chromatographic overlay of 0%, 0.001%, 0.003%, and 0.010% PS80 in 1.0 μg/ml Sample Solutions (T=0).

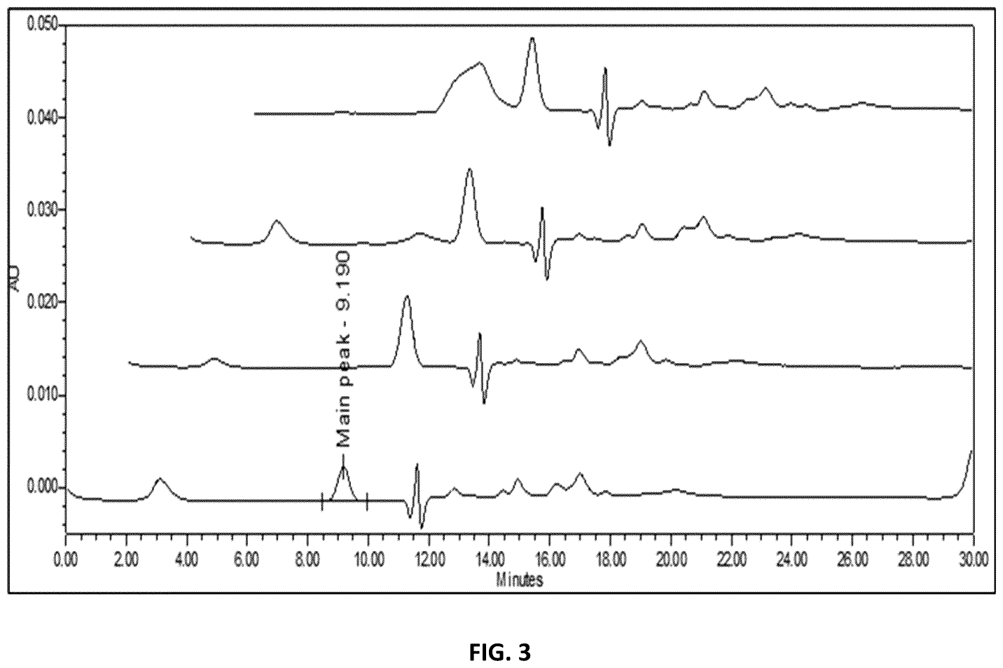

FIG. 4 shows a chromatographic overlay of 0%, 0.001%, 0.003%, and 0.010% PS80 in 1.0 μg/ml Sample Solutions (T=8).

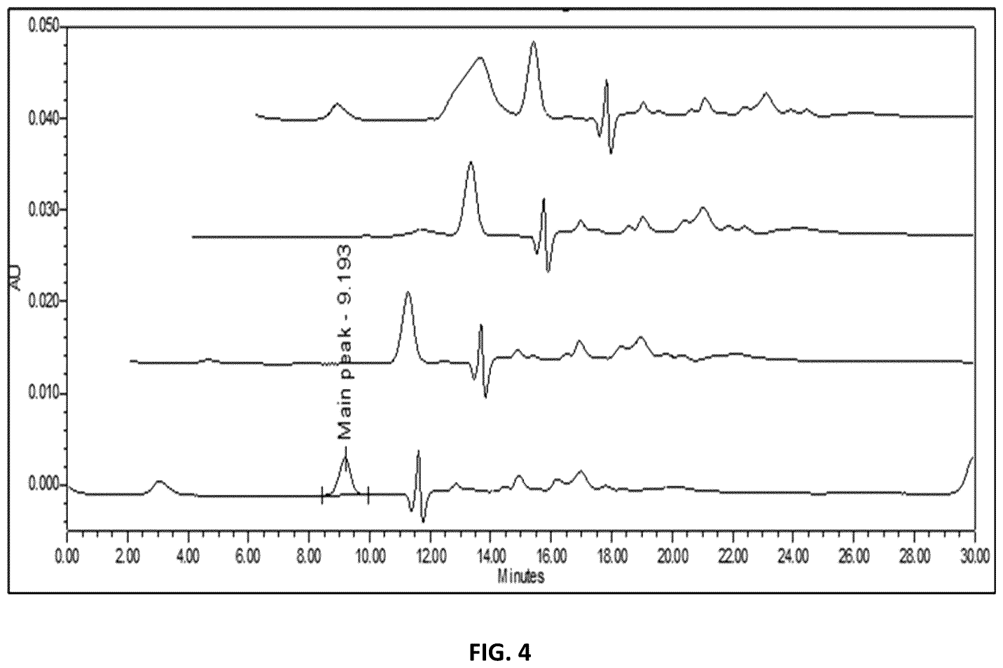

FIG. 5 shows a chromatographic overlay of 0%, 0.003%, 0.020%, and 0.100% PS20 in 0.5 μg/ml Sample Solutions (T=0).

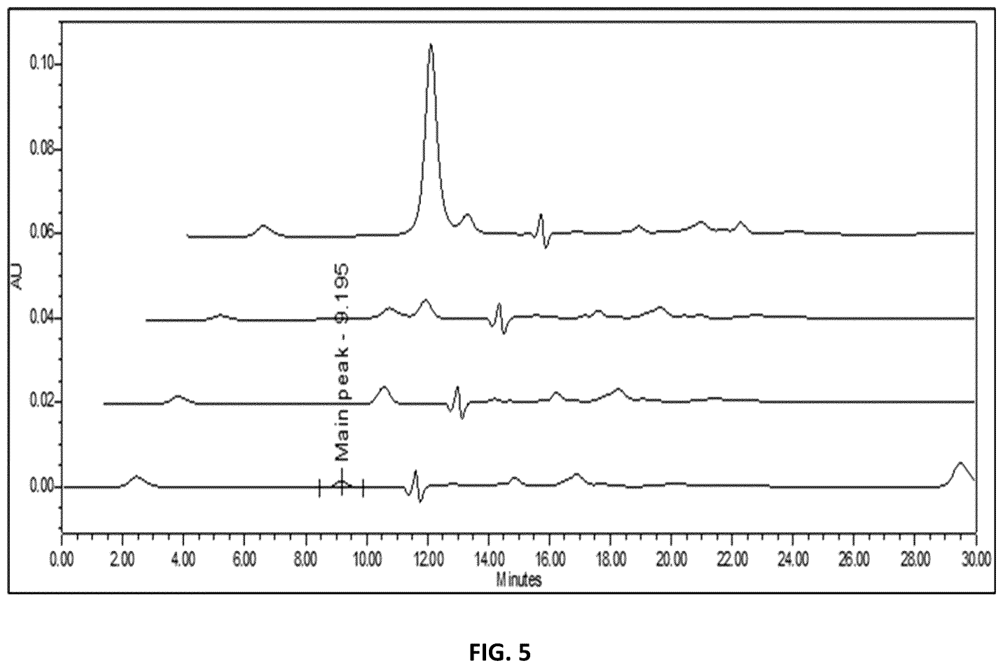

FIG. 6 shows a chromatographic overlay of 0%, 0.003%, 0.020%, and 0.100% PS20 in 0.5 μg/ml Sample Solutions (T=8).

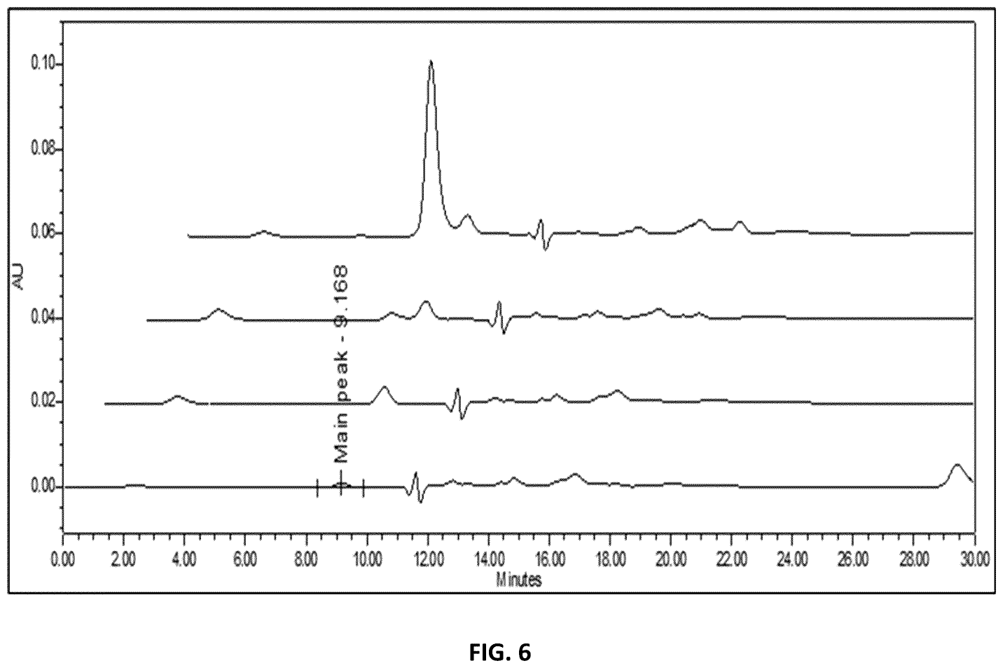

FIG. 7 shows a chromatographic overlay of 0%, 0.001%, 0.003%, and 0.010% PS80 in 0.5 μg/ml Sample Solutions (T=0).

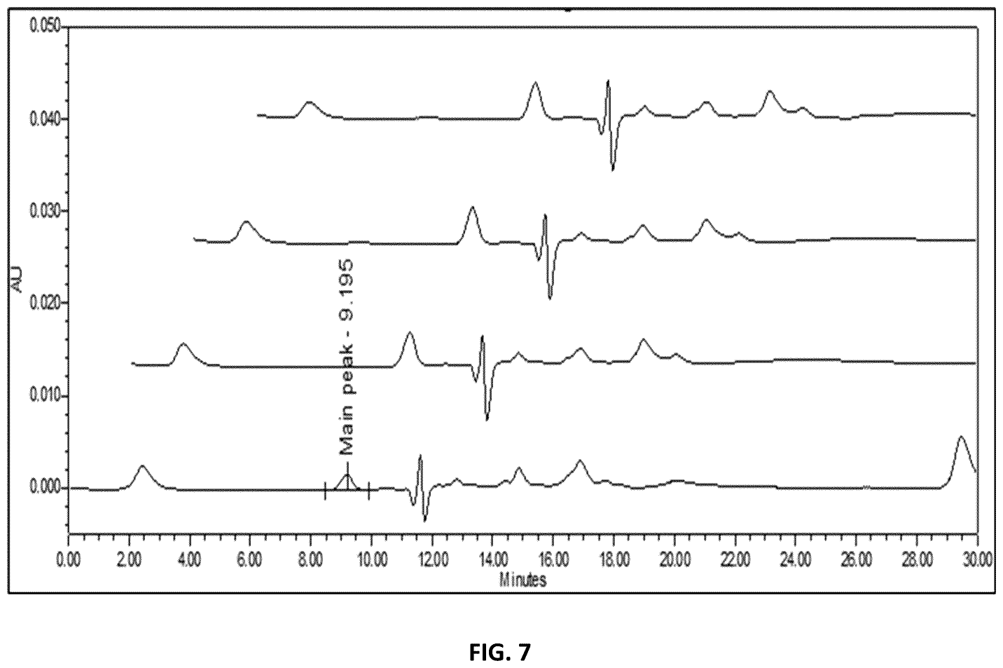

FIG. 8 shows a chromatographic overlay of 0%, 0.001%, 0.003%, and 0.010% PS80 in 0.5 μg/ml Sample Solutions (T=8).

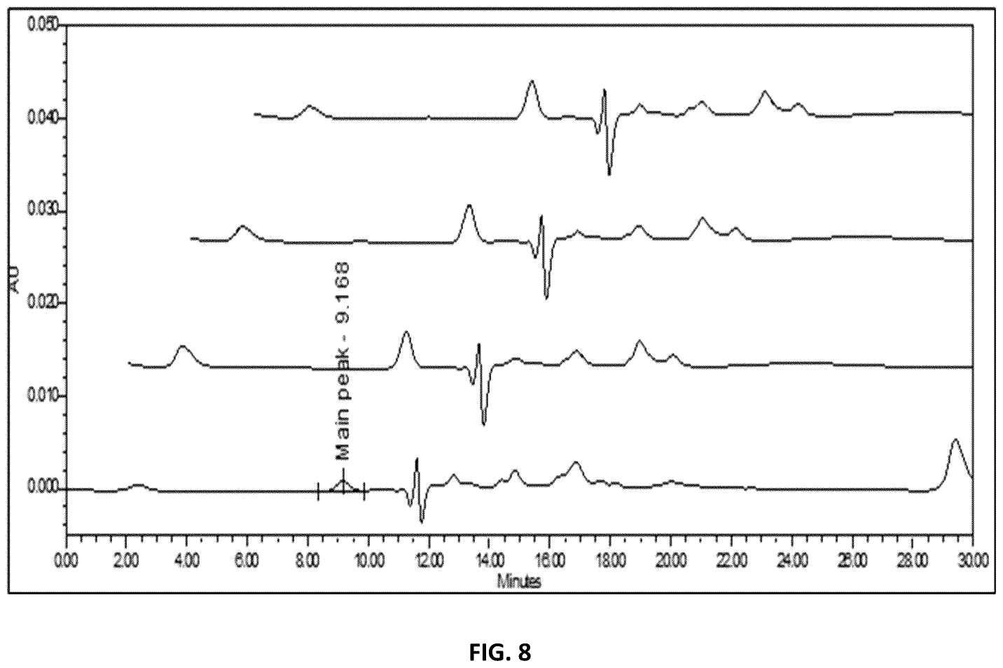

### DETAILED DESCRIPTION

Embodiments of the present disclosure relate generally to the discovery that polysorbate surfactant-based compositions and methods significantly reduce adsorption of KLK1 to polyolefin IV systems (including polyolefin IV bags, polyolefin IV lines, and polyolefin IV ports), improve overall recovery of KLK1, and thus allow for precise and consistent control of KLK1 dosages when administering to patients via IV infusion.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, chemical synthesis, and tissue culture, among others. Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below:

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

The term “composition” includes “pharmaceutical compositions”, “therapeutic compositions”, “solutions” such as IV solutions, “formulations”, and “dosage forms”.

The terms “endotoxin free” or “substantially endotoxin free” relate generally to compositions, solvents, devices, and/or vessels such as IV bags that contain at most trace amounts (e.g., amounts having no clinically adverse physiological effects to a subject) of endotoxin, and preferably undetectable amounts of endotoxin. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art. Also included are methods of producing KLK1 polypeptides in and isolating them from eukaryotic cells such as mammalian cells to reduce, if not eliminate, the risk of endotoxins being present in a composition of the invention. Preferred are methods of producing KLK1 polypeptides in and isolating them from recombinant cells grown in chemically defined, serum free media.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/ml, or EU/mg protein. Typically, 1 ng lipopolysaccharide (LPS) corresponds to about 1-10 EU.

The “half-life” of an agent such as a KLK1 polypeptide (e.g., DM199) can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the levels of agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulate” and “alter” include “increase,” “enhance” or “stimulate,” as well as “decrease”, “inhibit”, or “reduce,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount or level produced by a control composition, sample or test subject. A “decreased”, “inhibited, or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount or level produced a control composition, sample or test subject. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

A result is typically referred to as “statistically significant” if it is unlikely to have occurred by chance. The significance level of a test or result relates traditionally to the amount of evidence required to accept that an event is unlikely to have arisen by chance. In certain cases, statistical significance may be defined as the probability of deciding to reject the null hypothesis when the null hypothesis is actually true (a decision known as a Type I error, or “false positive determination”). This decision is often made using the p-value: if the p-value is less than the significance level, then the null hypothesis is rejected. The smaller the p-value, the more significant the result. Bayes factors may also be utilized to determine statistical significance (see Goodman, Ann Intern Med. 130:1005-13, 1999).

The term “solubility” refers to the property of a KLK1 polypeptide provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 6.0, pH 7.0, pH 7.4, pH 8.0 or pH 9.0. In certain embodiments, solubility is measured in water or a physiological buffer such as PBS or NaCl (with or without NaP). In specific embodiments, solubility is measured at relatively lower pH (for example, pH 6.0) and relatively higher salt (for example, 500 mM NaCl and 10 mM NaP). In certain embodiments, solubility is measured in a biological fluid (solvent) such as blood or serum. In certain embodiments, the temperature can be about room temperature (for example, about 20, about 21, about 22, about 23, about 24, or about 25° C.) or about body temperature (37° C.). In certain embodiments, a KLK1 polypeptide has a solubility of at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, or at least about 60 mg/ml at room temperature or at 37° C.

“Substantially” or “essentially” means nearly totally or completely, for instance, 95%, 96%, 97%, 98%, 99% or greater of some given quantity.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

As used herein, the terms “therapeutically effective amount”, “therapeutic dose,” “prophylactically effective amount,” or “diagnostically effective amount” is the amount of an agent such as a KLK1 polypeptide (e.g., DM199) needed to elicit the desired biological response following administration.

A “subject”, as used herein, includes any animal that exhibits a symptom, or is at risk for exhibiting a symptom, which can be treated with a KLK1 polypeptide or a dosage form thereof. Suitable subjects (patients) include laboratory animals (such as mouse, rat, rabbit, or guinea pig), farm animals, and domestic animals or pets (such as a cat or dog). Non-human primates and, preferably, human patients, are included.

By “isolated” is meant material that is substantially or essentially free from components that normally accompany it in its native state. For example, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances such as host cell proteins or nucleic acids.

A “wild type” or “reference” sequence or the sequence of a “wild type” or “reference” protein/polypeptide may be the reference sequence from which variant polypeptides are derived through the introduction of changes. In general, the “wild type” amino acid sequence for a given protein is the sequence that is most common in nature. Similarly, a “wild type” gene sequence is the polynucleotide sequence for that gene which is most commonly found in nature. Mutations can be introduced into a “wild type” gene (and thus the protein it encodes) either through natural processes or through human induced means.

Each embodiment in this specification is to be applied to every other embodiment unless expressly stated otherwise.

As disclosed herein, embodiments of the present disclosure relate generally to the discovery that polysorbate (PS) surfactant-based compositions and methods significantly reduce adsorption of KLK1 to polyolefin IV systems (including polyolefin IV bags, polyolefin IV lines, and polyolefin IV ports), improve overall recovery of KLK1, and thus allow for precise and consistent control of KLK1 dosages when administering to patients via IV infusion. Certain embodiments relate to a single vial approach, for example, a single vial with one or more KLK1 polypeptides (e.g., DM199) formulated with a PS surfactant such as PS20 or PS80. In these and related embodiments, the single vial is diluted into a polyolefin-containing IV system such as a polyolefin IV bag prior to the final IV solution being intravenously administered to a subject in need thereof. Some embodiments relate to a two file approach, for example, comprising a vial with one or more KLK1 polypeptides (e.g., DM199) and a separate vial with a PS surfactant such as PS20 or PS80. In some of these and related embodiments, the vial with the PS surfactant is first added to a polyolefin-containing IV system such as an polyolefin IV bag, and then the vial with the one or more KLK1 polypeptides (e.g., DM199) is separately added to same polyolefin-containing IV system, followed by appropriate inversion, agitation, or other means to disperse the one or more KLK1 polypeptides and the PS surfactant prior to the final IV solution being intravenously administered to a subject in need thereof.

The term “polysorbate” or “PS” surfactant refer to a family of amphipathic, nonionic surfactants that are derived from ethoxylated sorbitan or isosorbide (a derivative of sorbitol) esterified with fatty acids. “Polysorbate 20” or “PS20” includes a polysorbate-type nonionic surfactant with the IUPAC name “polyoxyethylene (20) sorbitan monolaurate”, a CAS Registry Number of 9005-64-5, and a chemical formula represented by C58H114026. Exemplary commercial sources include KOLLIPHOR PS 20, SCATTICS, ALKEST TW 20, TWEEN 20, and KOTILEN-20. In some embodiments, the PS20 chemical formula (C58H114026) accounts for about or at least about 20, 30, 40, 50, 60, 70, 80, or 90% of the total polysorbate in a given composition or formulation that comprises PS20. “Polysorbate 80” or “PS80” includes a polysorbate-type nonionic surfactant with the IUPAC name “polyoxyethylene (80) sorbitan monooleate”, CAS Registry Number of 9005-65-6, and a chemical formula represented by C64H124026. Exemplary commercial sources include KOLLIPHOR PS 80, ALKEST TW 80, SCATTICS, CANARCEL, POEGASORB 80, MONTANOX 80, TWEEN 80, and KOTILEN-80. In some embodiments, the PS80 chemical formula (C64H124026) accounts for about or at least about 20, 30, 40, 50, 60, 70, 80, or 90% of the total polysorbate in a given composition or formulation that comprises PS80.

For the single vial approach, certain embodiments include a pharmaceutical composition, comprising:
(i) one or more tissue kallikrein (KLK1) polypeptides (e.g., DM199); and
(ii) a PS surfactant, for example, such as PS20 or PS80.

In some embodiments, the one or more KLK1 polypeptides of (i) are at a concentration of about 10 μg/mL to about 2000 μg/mL, for example, a concentration of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1150, 1200, 1250, 1300, 1350, 1400, 1450, 1500, 1550, 1600, 1650, 1700, 1750, 1800, 1850, 1900, 1950, or 2000 μg/mL. In certain embodiments, the one or more KLK1 polypeptides of (i) are at a concentration of about 10-1000, 10-900, 10-800, 10-700, 10-600, 10-500, 10-400, 10-300, 10-200, 10-100, 10-90, 10-80, 10-70, 10-60, 10-50, 10-40, 10-30, or 10-20 μg/mL, or a concentration of about 20-1000, 20-900, 20-800, 20-700, 20-600, 20-500, 20-400, 20-300, 20-200, 20-100, 20-90, 20-80, 20-70, 20-60, 20-50, 20-40, or 20-30 g/mL, or a concentration of about 30-1000, 30-900, 30-800, 30-700, 30-600, 30-500, 30-400, 30-300, 30-200, 30-100, 30-90, 30-80, 30-70, 30-60, 30-50, 30-40 μg/mL, or a concentration of about 40-1000, 40-900, 40-800, 40-700, 40-600, 40-500, 40-400, 40-300, 40-200, 40-100, 40-90, 40-80, 40-70, 40-60, or 40-50 μg/mL, or a concentration of about 50-1000, 50-900, 50-800, 50-700, 50-600, 50-500, 50-400, 50-300, 50-200, 50-100, 50-90, 50-80, 50-70, or 50-60 μg/mL, or a concentration of about 60-1000, 60-900, 60-800, 60-700, 60-600, 60-500, 60-400, 60-300, 60-200, 60-100, 60-90, 60-80, or 60-70 μg/mL, or a concentration of about 70-1000, 70-900, 70-800, 70-700, 70-600, 70-500, 70-400, 70-300, 70-200, 70-100, 70-90, or 70-80 μg/mL, or a concentration of about 80-1000, 80-900, 80-800, 80-700, 80-600, 80-500, 80-400, 80-300, 80-200, 80-100, or 80-90 μg/mL, or a concentration of about 90-1000, 90-900, 90-800, 90-700, 90-600, 90-500, 90-400, 90-300, 90-200, or 90-100 μg/mL, or a concentration of about 100-1000, 100-900, 100-800, 100-700, 100-600, 100-500, 100-400, 100-300, 100-200 μg/mL, or a concentration of about 50-200, 60-200, 70-200, 80-200, 90-200, 100-200, 50-150, 60-150, 70-150, 80-150, 90-150, 100-150, 50-125, 60-125, 70-125, 80-125, 90-125, or 100-125 g/mL. In preferred embodiments, the one or more KLK1 polypeptides of (i) are at a concentration of about 50-150 g/mL, about 60-140 μg/mL, about 70-130 μg/mL, about 80-120 μg/mL, about 90-110 μg/mL, about 95-105 μg/mL, or about 100 μg/mL.

In particular embodiments, the PS surfactant of (ii) is at a concentration of about 0.01% to about 2.0%, for example, about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, or 2.0%. In preferred embodiments, the PS surfactant of (ii) is at a concentration of about 0.43% to about 0.87% to about 1.74% (for example, for use with a 50 mL or 100 mL IV bag), including about 0.40, 0.41, 0.42, 0.43, 0.44, 0.45, 0.46, 0.47, 0.48, 0.49, 0.50, 0.60, 0.70, 0.80, 0.81, 0.82, 0.83, 0.84, 0.85, 0.86, 0.87, 0.88, 0.89, 0.90, 0.95, 1.0, 1.1, 1.2, 1.3, 1.4, 1.5, 1.6, 1.7, 1.71, 1.72, 1.73, 1.74, 1.75, 1.76, 1.77, 1.78, 1.79, or 1.80%. In some embodiments, the PS surfactant of (ii) is PS20. In particular embodiments, the PS surfactant of (ii) is PS80.

In certain embodiments, the pharmaceutical composition is in a (single) vial at a total volume of about 0.1 mL to about 5 mL, for example, about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, or 5 mL. In preferred embodiments, the pharmaceutical composition is in a (single) vial at a total concentration of about 0.15 mL to about 0.5 mL. In specific pharmaceutical compositions, the one or more KLKL1 polypeptides of (i), for instance, DM199, are at concentration of about 100 μg/mL, the PS surfactant of (ii) is at a concentration of about 0.43% to about 0.87% (e.g., for use with 50 mL IV bag), and the composition is in a vial at a total volume of about 0.15 mL to about 0.5 mL. In some pharmaceutical compositions, the one or more KLKL1 polypeptides of (i), for instance, DM199, are at concentration of about 100 μg/mL, the PS surfactant of (ii) is at a concentration of about 0.43% to about 0.86% or up to about 1.74% (e.g., for use with 100 mL IV bag), and the composition is in a vial at a total volume of about 0.15 mL to about 0.5 mL.

In specific embodiments, as described herein, the one or more KLKL1 polypeptides of (i) comprise or consist of DM199.

In certain embodiments of the single vial approach, a pharmaceutical composition described herein is for use in a method of diluting the pharmaceutical composition into an IV solution in a polyolefin IV bag, including wherein the IV solution/polyolefin IV bag comprising the diluted pharmaceutical composition is for use in treating a disease or condition in a subject in need thereof. Also included is the use of a single vial pharmaceutical composition described herein in the preparation of a medicament for treating a disease or condition in a subject in need thereof, including wherein preparation of the medicament comprises diluting the pharmaceutical composition into an IV solution in a polyolefin IV bag. In certain of these and related embodiments, the final IV solution is for intravenous administration to the subject at a total KLK1 polypeptide intravenous dosage of about 0.1 μg/kg to about 0.25 μg/kg to about 0.5 μg/kg to about 5 μg/kg or to about 10.0 μg/kg. Exemplary diseases and conditions are described herein.

For the single vial approach, certain embodiments include methods of preparing a polyolefin IV bag for intravenous administration to a subject in need thereof, wherein the polyolefin IV bag comprises an IV solution, comprising:
(a) adding a (single vial) pharmaceutical composition described herein to the IV solution in the polyolefin IV bag; and
(b) inverting or agitating or otherwise mixing the polyolefin IV bag for a time sufficient to disperse the pharmaceutical composition into a final IV solution.

In some embodiments, the IV solution is a normal saline (0.9% sodium chloride), a half-strength normal saline (0.45% sodium chloride), a dextrose solution (for example, 1, 2, 3, 4, or 5% n water), or a Lactated Ringer's (LR) solution. In some embodiments, the polyolefin IV bag with IV solution has a total volume of about 50-1000 ml, for example, about 50 ml, about 100 ml, about 250 ml, about 500 ml, or about 1000 ml. In preferred embodiments, the polyolefin IV bag with IV solution has a total volume of about 50 or 100 mL. Polyolefin IV bags and IV solutions are known in the art and commercially-available.

In some embodiments, the single vial is added to or diluted into the IV solution in the polyolefin IV bag to create a “final IV solution”, which can then be intravenously administered to a subject in need thereof. In some embodiments, the one or more KLK1 polypeptides of (i), for example, DM199, are in the final IV solution at a concentration of about 0.03 μg/mL to about 15.0 μg/mL, including about 0.03, 0.04, 0.05, 0.06, 0.07, 0.08, 0.09, 0.10, 0.20, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 μg/mL. In preferred embodiments, the one or more KLK1 polypeptides of (i), for example, DM199, are in the final IV solution at a concentration about 0.15 μg/mL to about 1.0 μg/mL.

In some embodiments, the PS surfactant of is in the final IV solution at a concentration of about 0.0001% to about 0.10%, including about 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.10%. In preferred embodiments, the PS surfactant is in the final IV solution at a concentration of about 0.001% to about 0.002%. In some embodiments, the PS surfactant in the final IV solution is PS20. In particular embodiments, the PS surfactant in the final IV solution is PS80.

In specific embodiments, the pharmaceutical composition of step (a) comprises the one or more KLK1 polypeptides of (i), for example, DM199, at concentration of about 100 μg/mL, and the PS surfactant of (ii) at a concentration of about 0.43% to about 0.87%, wherein the polyolefin IV bag with the final IV solution of step (b) has a total volume of about 50 mL, the one or more KLK1 polypeptides, for example, DM199, are in the final IV solution at a concentration of about 0.15 μg/mL to about 1.0 μg/mL, and the PS surfactant is in the final IV solution at a concentration of about 0.001% to about 0.002%.

In particular embodiments, the pharmaceutical composition of step (a) comprises the one or more KLK1 polypeptides of (i), optionally DM199, at concentration of about 100 μg/mL, and the PS surfactant of (ii) at a concentration of about 0.43% to about 0.87% to about 1.74%, and wherein the polyolefin IV bag with the final IV solution of step (b) has a total volume of about 100 mL, wherein the one or more KLK1 polypeptides, for instance, DM199, are in the final IV solution at a concentration of about 0.15 μg/mL to about 1.0 μg/mL, and the PS surfactant is in the final IV solution at a concentration of about 0.001% to about 0.002%.

Certain embodiments comprising the step of (c) intravenously administering the final IV solution to the subject in need thereof via the polyolefin IV bag, for example, at a total KLK1 polypeptide intravenous dosage of about 0.1 μg/kg to about 0.25 μg/kg to about 0.5 μg/kg to about 5 μg/kg or to about 10.0 μg/kg. Also included are methods of treating a disease or condition in a subject in need thereof, comprising administering a final IV solution to the subject in need thereof via the polyolefin IV bag as prepared according to the foregoing description, for example, at a total KLK1 polypeptide intravenous dosage of about 0.1 μg/kg to about 0.25 μg/kg to about 0.5 μg/kg to about 5 μg/kg or to about 10.0 μg/kg. Exemplary diseases and conditions are described herein.

For the two vial approach, certain embodiments include a patient care kit, comprising:
(i) a first vial comprising a pharmaceutical composition, wherein the pharmaceutical composition comprises one or more tissue kallikrein (KLK1) polypeptides; and (ii) a second vial comprising a polysorbate surfactant, for instance, PS20 or PS80.

In certain embodiments, the one or more KLK1 polypeptides in the pharmaceutical composition of the first vial are at a concentration of about 10 μg/mL to about 500 μg/mL, for example, a concentration of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or 500 μg/mL. In certain embodiments, the one or more KLK1 polypeptides in the pharmaceutical composition of the first vial are at a concentration of about 10-500, 10-400, 10-300, 10-200, 10-100, 10-90, 10-80, 10-70, 10-60, 10-50, 10-40, 10-30, or 10-20 g/mL, or a concentration of about 20-500, 20-400, 20-300, 20-200, 20-100, 20-90, 20-80, 20-70, 20-60, 20-50, 20-40, or 20-30 μg/mL, or a concentration of about 30-500, 30-400, 30-300, 30-200, 30-100, 30-90, 30-80, 30-70, 30-60, 30-50, 30-40 μg/mL, or a concentration of about 40-500, 40-400, 40-300, 40-200, 40-100, 40-90, 40-80, 40-70, 40-60, or 40-50 μg/mL, or a concentration of about 50-500, 50-400, 50-300, 50-200, 50-100, 50-90, 50-80, 50-70, or 50-60 μg/mL, or a concentration of about 60-500, 60-400, 60-300, 60-200, 60-100, 60-90, 60-80, or 60-70 μg/mL, or a concentration of about 70-500, 70-400, 70-300, 70-200, 70-100, 70-90, or 70-80 μg/mL, or a concentration of about 80-500, 80-400, 80-300, 80-200, 80-100, or 80-90 μg/mL, or a concentration of about 90-500, 90-400, 90-300, 90-200, or 90-100 μg/mL, or a concentration of about 100-500, 100-400, 100-300, 100-200 μg/mL, or a concentration of about 50-200, 60-200, 70-200, 80-200, 90-200, 100-200, 50-150, 60-150, 70-150, 80-150, 90-150, 100-150, 50-125, 60-125, 70-125, 80-125, 90-125, or 100-125 μg/mL. In preferred embodiments, the one or more KLK1 polypeptides in the pharmaceutical composition of the first vial are at a concentration of about 50-150 μg/mL, about 60-140 μg/mL, about 70-130 μg/mL, about 80-120 μg/mL, about 90-110 μg/mL, about 95-105 μg/mL, or about 100 μg/mL.

In some embodiments, the PS surfactant in the second vial is at a concentration of about 0.005% to about 5.0%, or at a concentration of about 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.9, or 5.0%. In specific kits, the PS surfactant in the second vial is at a concentration of about 0.1% to about 0.2% to about 0.4% to about 0.8%. In some kits, the PS surfactant of (ii) is at a concentration of about 0.1% (for example, for use with 50 mL IV bag). In some embodiments, the PS surfactant of (ii) is at a concentration of about 0.2% (for example, for use with 100 mL IV bag). In some embodiments, the PS surfactant of (ii) is PS20. In some embodiments, the PS surfactant of (ii) is PS80.

In some kits, the first vial is at a total volume of about 0.10 mL to about 5.0 mL, for example, about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, or 5 mL; and the second vial is at a total volume of about 0.25 mL to about 0.5 mL to about 1.0 ml to about 2.0 mL, for example, about 0.25, 0.30, 0.35, 0.40, 0.45, 0.50, 0.55, 0.60, 0.65, 0.70, 0.75, 0.80, 0.85, 0.90, 0.95, 1.00, 1.10, 1.15, 1.25, 1.30, 1.35, 1.40, 1.45, 1.50, 1.55, 1.60, 1.65, 1.70, 1.75, 1.80, 1.85, 1.90, 1.95, or 2.0 mL, and in preferred embodiments, the second vial is at a total volume of about 1 mL.

In specific kits, the first vial is at a total volume of about 0.15 mL to about 0.5 mL and comprises the pharmaceutical composition of one or more KLK1 polypeptides, for instance, DM199, at a concentration of about 100 μg/mL, and the second vial is at a total volume of about 0.25 mL to about 2.0 mL and comprises the polysorbate surfactant at a concentration of about 0.1% to about 0.8%, including wherein the second vial is about 2 mL total volume/about 0.1% surfactant, about 1 mL total volume/about 0.2% surfactant, about 0.5 mL total volume/about 0.4% surfactant, or about 0.25 mL total volume/about 0.8% surfactant.

In some patient care kits, the pharmaceutical composition of (i) comprises or consists of DM199.

Certain patient care kits are for use in a method of diluting the first vial and the second vial into an IV solution in a polyolefin IV bag, including wherein the IV solution/polyolefin IV bag comprising the diluted first and second vials is for use in treating a disease or condition in a subject in need thereof. Also included is the use of a patient care kit described herein in the preparation of a medicament for treating a disease or condition in a subject in need thereof, wherein preparation of the medicament comprises diluting the first vial and the second vial into an IV solution in a polyolefin IV bag. Exemplary disease and conditions are described herein.

Also for the two vial approach, certain embodiments include methods of preparing a polyolefin intravenous (IV) bag for intravenous administration to a subject in need thereof, wherein the polyolefin IV bag comprises an IV solution, comprising:

(a) adding a polysorbate surfactant to the IV solution in the IV bag, including wherein the polysorbate surfactant is selected from PS20 and PS80;
(b) separately adding a pharmaceutical composition comprising one or more tissue kallikrein (KLK1) polypeptides to the IV solution in the polyolefin IV bag; and
(c) inverting or agitating or otherwise mixing the IV bag for a time sufficient to disperse the pharmaceutical composition and the polysorbate surfactant into a final IV solution.

Steps (a) and (b) can be performed in any order. Typically, step (a) is performed first to minimize adsorption of the KLK1 polypeptides to the polyolefin IV bag. Examples of IV solutions, as described herein, include normal saline (0.9% sodium chloride), a half-strength normal saline (0.45% sodium chloride), a dextrose solution (1, 2, 3, 4, or 5% in water), or a Lactated Ringer's (LR) solution. In certain embodiments, the polyolefin IV bag with IV solution has a total volume of about 50-1000 ml, including about 50 ml, about 100 ml, about 250 ml, about 500 ml, or about 1000 ml. In preferred embodiments, the polyolefin IV bag with IV solution has a total volume of about 50 mL or 100 mL.

In some embodiments, the PS surfactant in (a) is added to the final IV solution of (c) at a concentration of about 0.0001% to about 0.10%, including about 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.10%. In preferred embodiments, the PS surfactant is added to the final IV solution at concentration of about 0.001% to about 0.002%. In some embodiments, the PS surfactant in the final IV solution is PS20. In some embodiments, the PS surfactant in the final IV solution is PS80.

In certain of the methods for the two vial approach, the one or more KLK1 polypeptides in (b), for example, DM199, are added to the final IV solution of (c) to achieve a concentration of about 0.03 μg/mL to about 15.0 μg/mL, including about 0.03, 0.04, 0.05, 0.06, 0.07, 0.08, 0.09, 0.10, 0.20, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 μg/mL. In preferred embodiments, the one or more KLK1 polypeptides of (i), for example, DM199, are added to the final IV solution of (c) to achieve a concentration about 0.15 μg/mL to about 1.0 μg/mL.

In certain of the methods for the two vial approach, the polyolefin IV bag with the final IV solution of (c) has a total volume about 50 ml or 100 mL, the polysorbate surfactant added from (a) is in the final IV solution at a concentration of about 0.001% to about 0.002%, and the one or more KLK1 polypeptides added from (b), for instance, DM199, are in the final IV solution at a concentration of about 0.15 μg/mL to about 1.0 μg/mL. Certain of the methods for the two vial approach are performed using a patient care kit described herein.

Certain methods comprise the step of (d) intravenously administering the final IV solution to the subject in need thereof via the polyolefin IV bag, for example, at a total KLK1 polypeptide intravenous dosage of about 0.1 μg/kg to about 0.25 μg/kg to about 0.5 μg/kg to about 5 μg/kg or to about 10.0 μg/kg. Also included are methods of treating a disease or condition in a subject in need thereof, comprising administering a final IV solution to the subject in need thereof via the polyolefin IV bag as prepared according to the methods described herein, for example, at a total KLK1 polypeptide intravenous dosage of about 0.1 μg/kg to about 0.25 μg/kg to about 0.5 μg/kg to about 5 μg/kg or to about 10.0 μg/kg.

Certain embodiments relate generally to polyolefin-containing IV systems such as polyolefin IV bags, which are prepared according to the pharmaceutical compositions, patient care kits, and/or methods described herein. For example, certain embodiments include a polyolefin IV bag, comprising a final IV solution of:
(i) one or more tissue kallikrein (KLK1) polypeptides; and
(ii) a polysorbate surfactant selected from PS20 and PS80.

In certain polyolefin IV bags, the IV solution is a normal saline (0.9% sodium chloride), a half-strength normal saline (0.45% sodium chloride), a dextrose solution (for example, 1, 2, 3, 4, or 5% in water), or a Lactated Ringer's (LR) solution. In some embodiments, IV bag with solution has a total volume of about 50-1000 ml, for example, about 50 ml, about 100 ml, about 250 ml, about 500 ml, or about 1000 ml. In certain IV bags, the more KLK1 polypeptides of (i), for example, DM199, are in the final IV solution at a concentration of about 0.03 μg/mL to about 15.0 μg/mL, including about 0.03, 0.04, 0.05, 0.06, 0.07, 0.08, 0.09, 0.10, 0.20, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 μg/mL. In preferred IV bags, the more KLK1 polypeptides of (i), for example, DM199, are in the final IV solution at a concentration of about 0.15 μg/mL to about 1.0 μg/mL.

In some polyolefin IV bags, the PS surfactant of (ii) is in the final IV solution at a concentration of about 0.0001% to about 0.10%, including about 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.10%. In preferred polyolefin IV bats, the PS surfactant of (ii) is in the final IV solution at a concentration of about 0.001% to about 0.002%. In some IV bags, the PS surfactant of (ii) is PS20. In certain IV bags, the PS surfactant of (ii) is PS80.

In particular polyolefin IV bags, the final IV solution has a total volume about 50 ml or 100 mL, the one or more KLK1 polypeptides from (i), for instance, DM199, are in the final IV solution at a concentration of about 0.15 μg/mL to about 1.0 μg/mL, and the PS surfactant from (ii) is in the final IV solution at a concentration of about 0.001% to about 0.002%. In specific IV bags, the one or more KLK polypeptides of (i) comprise or consist of DM199.

In some embodiments, the polyolefin IV bags described herein are for use in treating a disease or condition in a subject in need thereof, for example, by IV administration at a total KLK1 polypeptide intravenous dosage of about 0.1 μg/kg to about 0.25 μg/kg to about 0.5 μg/kg to about 5 μg/kg or to about 10.0 μg/kg. Also included is the use of the polyolefin IV bags described herein in the preparation of a medicament for treating a disease or condition in a subject in need thereof, for instance, by IV administration at a total KLK1 polypeptide intravenous dosage of about 0.1 μg/kg to about 0.25 μg/kg to about 0.5 μg/kg to about 5 μg/kg or to about 10.0 μg/kg. Exemplary disease and conditions are described herein.

Tissue Kallikrein-1 (KLK1) Polypeptides. The compositions, patient care kits, methods, and related embodiments described herein comprise “one or more tissue kallikrein-1 (KLK1) polypeptides”. Tissue kallikrein polypeptides are members of a gene super family of serine proteases comprising at least 15 separate and distinct proteins (named tissue kallikrein 1 through 15) (Yousef et al., 2001, Endocrine Rev; 22:184-204). Tissue kallikrein-1 is a trypsin-like serine protease. In humans and animal tissues, tissue kallikrein-1 cleaves kininogen into lysyl-bradykinin (also known as kallidin), a decapeptide kinin having physiologic effects similar to those of bradykinin. Bradykinin is a peptide that causes blood vessels to dilate and therefore causes blood pressure to lower. Kallidin is identical to bradykinin with an additional lysine residue added at the N-terminal end and signals through the bradykinin receptor.

The KLK1 gene encodes a single pre-pro-enzyme that is 262 amino acid residues in length and that includes the “pre-” sequence (residues 1-18) and the “pro-” sequence (residues 19-24), which is activated by trypsin-like enzymes. The “mature” and “active” form human KLK1 is a glycoprotein of about 238 amino acid residues (residues 25-262) with a molecular weight of 26 kDa and a theoretical pI of 4.6. KLK1 has five disulfide bonds in its tertiary structure that are believed to be responsible for the protein's high stability, both against trypsin digestion and heat inactivation.

The amino acid sequence of full-length tissue kallikrein-1 is available for a wide variety of species, including, but not limited to, human (SEQ ID NO: 1 and SEQ ID NO: 2), mouse (see, for example, GenBank: AAA39349.1, Feb. 1, 1994); domestic cat (see, for example, NCBI Reference Sequence: XP_003997527.1, Nov. 6, 2012); gorilla (see, for example, NCBI Reference Sequence: XP_004061305.1, Dec. 3, 2012); cattle (see, for example, GenBank: AAI51559.1, Aug. 2, 2007); dog (see, for example, CBI Reference Sequence: NP_001003262.1, Feb. 22, 2013); rat (see, for example, GenBank: CAE51906.1, Apr. 25, 2006); and olive baboon (see, for example, NCBI Reference Sequence: XP_003916022.1, Sep. 4, 2012). KLK1 is functionally conserved across species in its capacity to release the vasoactive peptide, Lys-bradykinin, from low molecular weight kininogen. A tissue kallikrein-1 polypeptide of the present invention may have any of the known amino acid sequences for KLK1, or a fragment or variant thereof.

In certain embodiments, a KLK1 polypeptide is a “mature” KLK1 polypeptide. In certain embodiments, a KLK1 polypeptide is a human KLK1 polypeptide, optionally a mature human KLK1 polypeptide. In particular embodiments, a KLK1 polypeptide is a recombinant human polypeptide, for example, a recombinant human KLK1 polypeptide, optionally in the mature form. Recombinant human KLK1 (rhKLK1) can provide certain advantages over other sources of KLK1, such as urinary KLK1 (e.g., human KLK1 isolated from human urine), including a homogenous preparation of rhKLK1, simpler regulatory path to licensure, and options to alter the amino acid sequence or glycosylation pattern based on cell culture conditions.

Exemplary amino acid sequences of human tissue kallikrein-1 (hKLK1) polypeptides are provided in Table K1 below.

TABLE K1
Exemplary KLK1 Sequences

| | | SEQ ID |
|---|---|---|
| Source | Sequence | NO: |
| Human | MWFLVLCLALSLGGTGAAPPIQSRIVGGWECEQHSQPWQAALYHFSTFQCGGILV | 1 |
| KLK1 | HRQWVLTAAHCISDNYQLWLGRHNLFDDENTAQFVHVSESFPHPGFNMSLLENHT | |
| | RQADEDYSHDLMLLRLTEPADTITDAVKVVELPTEEPEVGSTCLASGWGSIEPEN | |
| | FSFPDDLQCVDLKILPNDECKKAHVQKVTDFMLCVGHLEGGKDTCVGDSGGPLMC | |
| | DGVLQGVTSWGYVPCGTPNKPSVAVRVLSYVKWIEDTIAENS | |
| Human | MWFLVLCLALSLGGTGAAPPIQSRIVGGWECEQHSQPWQAALYHFSTFQCGGILV | 2 |
| KLK1 | HRQWVLTAAHCISDNYQLWLGRHNLFDDENTAQFVHVSESFPHPGFNMSLLENHT | |
| variant | RQADEDYSHDLMLLRLTEPADTITDAVKVVELPTQEPEVGSTCLASGWGSIEPEN | |
| | FSFPDDLQCVDLKILPNDECKKVHVQKVTDFMLCVGHLEGGKDTCVGDSGGPLMC | |
| | DGVLQGVTSWGYVPCGTPNKPSVAVRVLSYVKWIEDTIAENS | |
| Human | IVGGWECEQHSQPWQAALYHFSTFQCGGILVHRQWVLTAAHCISDNYQLWLGRHN | 3 |
| mature | LFDDENTAQFVHVSESFPHPGFNMSLLENHTRQADEDYSHDLMLLRLTEPADTIT | |
| KLK1 | DAVKVVELPTEEPEVGSTCLASGWGSIEPENFSFPDDLQCVDLKILPNDECKKAH | |
| | VQKVTDFMLCVGHLEGGKDTCVGDSGGPLMCDGVLQGVTSWGYVPCGTPNKPSVA | |
| | VRVLSYVKWIEDTIAENS | |
| Human | IVGGWECEQHSQPWQAALYHFSTFQCGGILVHRQWVLTAAHCISDNYQLWLGRHN | 4 |
| mature | LFDDENTAQFVHVSESFPHPGFNMSLLENHTRQADEDYSHDLMLLRLTEPADTIT | |
| KLK1 | DAVKVVELPTQEPEVGSTCLASGWGSIEPENFSFPDDLQCVDLKILPNDECKKVH | |
| variant | VQKVTDFMLCVGHLEGGKDTCVGDSGGPLMCDGVLQGVTSWGYVPCGTPNKPSVA | |
| | VRVLSYVKWIEDTIAENS | |

In certain embodiments, a KLK1 polypeptide comprises, consists, or consists essentially of SEQ ID NO: 1-3 or 4, or residues 1-262, residues 19-262, or residues 25-262 of SEQ ID NO: 1 or SEQ ID NO: 2, including fragments and variants thereof. Amino acids 1 to 18 of SEQ ID NO: 1 and 2 represent the signal peptide, amino acids 19 to 24 represent propeptide sequences, and amino acids 25 to 262 represent the mature peptide. Thus, the preproprotein includes a presumptive 17-amino acid signal peptide, a 7-amino acid proenzyme fragment and a 238-amino acid mature KLK1 protein.

A comparison between SEQ ID NO: 1 and SEQ ID NO: 2 (or SEQ ID NO: 3 and SEQ ID NO: 4) shows two amino acid differences between the two hKLK1 amino acid sequences. Single-nucleotide polymorphism (SNPs) between the two individuals within a species account for an E to Q substitution at amino acid residue 145 of 262 and an A to V substitution at position 188 of 262. SEQ ID NO: 1 has an E (glutamic acid) at position 145 and an A (alanine) at position 188, while SEQ ID NO: 2 has a Q (glutamine) at position 145 and a V (valine) at position 188. In some embodiments, KLK1 polypeptide has an E at position 145; a Q at position 145; an A at position 188; an A at position 188; an E at position 145 and an A at position 188; a Q at position 145 and a V at position 188; a Q at position 145 and an A at position 188; or an E at position 145 and a V at position 188.

As noted above, certain embodiments include active variants and fragments of a reference KLK1 polypeptide. A “variant” of a starting or reference polypeptide is a polypeptide that has an amino acid sequence different from that of the starting or reference polypeptide. Such variants include, for example, deletions from, insertions into, and/or substitutions of residues within the amino acid sequence of the polypeptide of interest. A variant amino acid, in this context, refers to an amino acid different from the amino acid at the corresponding position in a starting or reference polypeptide sequence. Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

In some embodiments, a KLK polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to a reference sequence, such as, for example, an amino acid sequence described herein (for example, SEQ ID NOs: 1-4).

In some aspects, a KLK1 polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to SEQ ID NO: 1 or 3, or to a fragment of SEQ ID NO: 1 or 3, such as for example, residues 25-262 or residues 78-141 of SEQ ID NO: 1. Such a KLK1 polypeptide may have an E or a Q at amino acid residue 145, and/or an A or a V at position 188.

In some aspects, a KLK1 polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to SEQ ID NO: 2 or 4, or to a fragment of SEQ ID NO: 2 or 4, such as for example, residues 25-262 or residues 78-141 of SEQ ID NO: 2. Such a KLK1 polypeptide may have an E or a Q at amino acid residue 145, and/or an A or a V at position 188.

“Percent (%) amino acid sequence identity” with respect to a polypeptide is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, California.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Variants may also include heterologous sequences or chemical modifications which are added to the reference KLK1 polypeptide, for example, to facilitate purification, improve metabolic half-life, or make the polypeptide easier to identify. Examples include affinity tags such as a His-tag, Fc regions, and/or a PEGylation sequence and PEG.

The term “fragment” includes smaller portions of a KLK1 polypeptide (or variants thereof) that retain the activity of a KLK1 polypeptide. Fragments includes, for example, a KLK1 polypeptide fragment that ranges in size from about 20 to about 50, about 20 to about 100, about 20 to about 150, about 20 to about 200, or about 20 to about 250 amino acids in length. In other embodiments, a KLK1 polypeptide fragment ranges in size from about 50 to about 100, about 50 to about 150, about 50 to about 200, or about 50 to about 250 amino acids in length. In other embodiments, a KLK1 polypeptide fragment ranges in size from about 100 to about 150, about 100 to about 200, about 100 to about 250, about 150 to about 175, about 150 to about 200, or about 150 to about 250 amino acids in length. In other illustrative embodiments, a KLK1 polypeptide fragment ranges in size from about 200 to about 250 amino acids in length. Certain embodiments comprise a polypeptide fragment of a full-length KLK1 of about, up to about, or at least about 50, 60, 70, 80, 90, 100, 110, 120, 130, 140, 150, 160, 170, 180, 190, 200, 210, 220, 230, 240, 250 or more (e.g., contiguous) amino acid residues. In some embodiments, a fragment may have residues 25-262 or residues 78-141 of a preproprotein sequence. In some embodiments, a fragment may be any such fragment size, as described above, of SEQ ID NO: 1 or SEQ ID NO: 2.

In some instances, fragments and variants of a KLK1 polypeptide retain the enzymatic capacity to release the vasoactive peptide, Lys-bradykinin, from low molecular weight kininogen. In some embodiments, an active variant or fragment retains serine protease activity of a KLK1 polypeptide that releases kallidin from a higher molecular weight precursor such as kininogen, or that cleaves a substrate similar to kininogen such as D-val-leu-arg-7 amido-4-trifluoromethylcoumarin to release a colorimetric or fluorometric fragment. The protease activity of KLK1 polypeptides can be measured in an enzyme activity assay by measuring either the cleavage of low-molecular-weight kininogen, or the generation of lys-bradykinin. In one assay format, a labeled substrate is reacted with the KLK1 glycoform, and the release of a labeled fragment is detected. One example of such a fluorogenic substrate suitable for KLK1 measurement of activity is D-val-leu-arg-7 amido-4-trifluoromethylcoumarin (D-VLR-AFC, FW 597.6) (Sigma, Cat #V2888 or Ana Spec Inc Cat #24137). When D-VLR-AFC is hydrolyzed, the free AFC produced in the reaction can be quantified by fluorometric detection (excitation 400 nm, emission 505 nm) or by spectrophotometric detection at 380 nm (extinction coefficient=12,600 at pH 7.2). Other methods and substrates may also be used to measure KLK1 proteolytic activity.

Glycoforms and Mixtures Thereof. In certain embodiments, the “one or more KLK1 polypeptides” described herein refers to a mixture of KLK1 polypeptide glycoforms, including compositions and related embodiments that comprise defined ratios of double and triple glycosylated KLK1 polypeptides (see, for example, U.S. Application No. 2015/0196624, incorporated by reference in its entirety).

Human kallikrein has three potential Asn-linked (N-linked) glycosylation sites at residues 78, 84, and 141, relative to the mature amino acid sequence shown, for example, in SEQ ID NO: 3 or 4, as well as putative O-linked glycosylation sites. However, O-linked glycosylation is not detected in naturally-occurring KLK1. By SDS-PAGE analysis, KLK1 polypeptides glycosylated at all three positions (positions 78, 84, and 141) are detected as the high molecular weight band and are referred to herein as the high-molecular weight, triple glycosylated glycoform of KLK1 (or “high glycoform” or “triple glycoform” KLK1). By SDS-PAGE analysis, KLK1 polypeptides glycosylated at only two of three available positions (positions 78 and 84) are detected as a low molecular weight band and are referred to herein as the low-molecular weight, double glycosylated glycoform of KLK1 (or as “low glycoform” or “double glycoform” KLK1).

Certain compositions therefore comprise a mixture of KLK1 glycoforms at a defined ratio, for example, comprising a first KLK1 polypeptide and a second KLK1 polypeptide, wherein the first KLK1 polypeptide has three glycans attached at the three different positions available for glycosylation in the polypeptide, and wherein the second KLK1 polypeptide has two glycans attached at only two of the three different positions available for glycosylation in the polypeptide. In certain embodiments, the first and second KLK1 polypeptides are present at a ratio of about 45:55 to about 55:45, including, for example, about 46:54, about 47:53, about 48:52, about 49:51, about 51:49, about 52:48, about 53:47, and about 54:46, including all integers and decimal points in between. In specific embodiments, the first and second KLK1 polypeptides are present at a ratio of about 50:50. In some embodiments, the ratio of the first and second KLK1 polypeptides is not about 60:40. In some embodiments, the ratio of the first and second KLK1 polypeptides is not about 40:60. In certain embodiments, the composition is free or substantially free of other glycosylated isoforms (glycoforms) of KLK1.

Some compositions comprise a triple glycoform of a KLK1 polypeptide and a double glycoform of a KLK1 polypeptide, wherein the triple glycoform and the double glycoform are present at a ratio of about 45:55 to about 55:45 including, for example, about 46:54, about 47:53, about 48:52, about 49:51, about 51:49, about 52:48, about 53:47, and about 54:46. In some embodiments, the triple glycoform and the double glycoform are present at a ratio of about 50:50. In some embodiments, the ratio of the triple glycoform and double glycoform is not about 60:40. In some embodiments, the ratio of the triple glycoform and double glycoform is not about 40:60. In certain embodiments, the composition is free or substantially free of other glycosylated isoforms (glycoforms) of KLK1.

In specific embodiments, the one or more KLK1 polypeptides, or mixture of KLK1 polypeptide glycoforms, is “DM199”. As used herein, “DM199” refers to a formulation composed of two glycoforms of a mature, human KLK1 variant polypeptide, each glycoform having the amino acid sequence set forth in SEQ ID NO: 4 (or amino acid residues 25-262 of SEQ ID NO: 2): one being a triple glycoform that has three N-linked glycans attached at residues 78, 84, and 141, and the other being a double glycoform that has two N-linked glycans attached at residues 78 and 84 but not 141 (the numbering being defined by SEQ ID NO: SEQ ID NO: 3 or 4), wherein the triple glycoform and the double glycoform are formulated at a ratio of about 50:50.

The ratios of the double and triple glycosylated isoforms of KLK1 can be detected and quantitated by a variety of methods, including high performance liquid chromatography (HPLC), which may include reversed phase (RP-HPLC), lectin affinity chromatography and lectin affinity electrophoresis. The preparation and characterization of KLK1 glycoform mixtures such as DM199 is described in U.S. Application No. 2015/0196624, incorporated by reference in its entirety.

Purity. In some embodiments, the “purity” of a composition (for example, formulation, solution, dosage form) is characterized, for example, by the amount (e.g., total amount, relative amount, percentage) of host cell protein(s), host cell DNA, endotoxin, and/or percentage single peak purity by SEC HPLC. In some instances, the purity of a composition is characterized by the amount (e.g., percentage) of KLK1 polypeptide relative to other components, for example, any one or more of the foregoing.

In some embodiments, the purity of a composition is characterized relative to or by the levels or amount of host cell proteins. The host cells used for recombinant expression may range from bacteria and yeast to cell lines derived from mammalian or insect species. The cells contain hundreds to thousands of host cell proteins (HCPs) and other biomolecules that could contaminate the final product. The HCP may be secreted along with the protein of interest, or released by accidental lysing of the cells, and may contaminate the protein of interest. Two types of immunological methods may be applied to HCP analysis: Western blotting (WB) and immunoassay (IA), which includes techniques such as ELISA and sandwich immunoassay or similar methods using radioactive, luminescent, or fluorescent reporting labels. Compositions of the present invention may include host cell protein of less than about 500, less than about 400, less than about 300, less than about 200, less than about 100 or less than about 50 ng/mg total protein.

In some instances, purity is characterized relative to or by the levels or amount of host cell DNA. Detection of residual host cell DNA may be performed by Polymerase Chain Reaction (PCR) with a variety of primers for sequences in the host cell genome. Residual host cell DNA is generally reported as being below a certain threshold level, but may also be quantitated with a rPCR method. Compositions of the present invention may include host cell deoxyribonucleic acid (DNA) of less than about 100, less than about 90, less than about 80, less than about 70, less than about 60, less than about 50, less than about 40, less than about 30, less than about 20, or less than about 10 pg/mg total protein.

In certain embodiments, purity is characterized relative to or by the amount or levels of endotoxin. As noted herein, endotoxin is extremely potent, heat stable, passes sterilizing membrane filters, and is present everywhere bacteria are or have been present. An Endotoxin Unit (EU) is a unit of biological activity of the USP Reference Endotoxin Standard.

The bacterial endotoxins test (BET) is a test to detect or quantify endotoxins from Gram-negative bacteria using amoebocyte lysate (white blood cells) from the horseshoe crab (Limulus polyphemus or Tachypleus tridentatus). Limulus amoebocyte lysate (LAL) reagent, FDA approved, is used for all USP endotoxin tests. There are at least three methods for this test: Method A, the gel-clot technique, which is based on gel formation; Method B, the turbidimetric technique, based on the development of turbidity after cleavage of an endogenous substrate;
and Method C, the chromogenic technique, based on the development of color after cleavage of a synthetic peptide-chromogen complex.

At least two types of endotoxin tests are described in the USP <85> BET. Photometric tests require a spectrophotometer, endotoxin-specific software and printout capability. The simplest photometric system is a handheld unit employing a single-use LAL cartridge that contains dried, pre-calibrated reagents; there is no need for liquid reagents or standards. The FDA-approved unit is marketed under the name of Endosafe®-PTS™. The device requires about 15 minutes to analyze small amounts of sample, a 25 μL aliquot from CSP diluted in a sterile tube, and to print out results. In contrast, gel-clot methods require a dry-heat block, calibrated pipettes and thermometer, vortex mixer, freeze-dried LAL reagents, LAL Reagent Water (LRW) for hydrating reagents and depyrogenated glassware. In this clot test, diluted sample and liquid reagents require about an hour for sample and positive-control preparation and an hour's incubation in a heat block; results are recorded manually. Thus, the simplicity and speed of the automated system make it ideally suited to the pharmacy setting.

In some instances, the purity of a composition is characterized by the degree of aggregation. For instance, the degree of aggregation of KLK1 can be determined by Size-exclusion chromatography (SEC), which separates particles on the basis of size. It is a generally accepted method for determining the tertiary structure and quaternary structure of purified proteins. SEC is used primarily for the analysis of large molecules such as proteins or polymers. SEC works by trapping these smaller molecules in the pores of a particle. The larger molecules simply pass by the pores as they are too large to enter the pores. Larger molecules therefore flow through the column quicker than smaller molecules, that is, the smaller the molecule, the longer the retention time. Certain embodiments are also substantially free of aggregates (greater than about 95% appearing as a single peak by SEC HPLC). Certain embodiments are free of aggregates with greater than about 96%, about 97%, about 98%, or about 99%, appearing as a single peak by SEC HPLC.

In certain embodiments, the “purity” of the KLK1 polypeptide(s) in a composition is specifically defined. For instance, certain embodiments comprise one or more hKLK1 polypeptides that are at least about 80, at least about 85, at least about 90, at least about 91, at least about 92, at least about 93, at least about 94, at least about 95, at least about 96, at least about 97, at least about 98, at least about 99, or 100% pure, including all decimals in between, relative to other components. Purity can be measured, for example and by no means limiting, by high performance liquid chromatography (HPLC), a well-known form of column chromatography used frequently in biochemistry and analytical chemistry to separate, identify, and quantify compounds.

In certain embodiments, a composition has one or more of the following determinations of purity: less than about 1 EU endotoxin/mg protein, less that about 100 ng host cell protein/mg protein, less than about 10 pg host cell DNA/mg protein, and/or greater than about 95% single peak purity by SEC HPLC.

In some instances, a composition is formulated with pharmaceutically acceptable excipients, diluents, adjuvants, or carriers, for instance, to optimize stability and achieve isotonicity. In certain aspects, the pH is near physiological pH or about pH 7.4, including about pH 6.5, about 7.0, about 7.1, about 7.2, about 7.3, about 7.4, about 7.5, about 7.6, about 7.7, about 7.8, about 7.9, about 8.0, about 8.5, or any range thereof. In some embodiments, a composition comprises a KLK1 polypeptide in combination with a physiologically acceptable carrier. Such carriers include pharmaceutically acceptable carriers, excipients, or stabilizers which are nontoxic to the cell or mammal being exposed thereto at the dosages and concentrations employed. Methods of formulation are well known in the art and are disclosed, for example, in Remington: The Science and Practice of Pharmacy, Mack Publishing Company, Easton, Pa., Edition 21 (2005).

The phrase “physiologically-acceptable” or “pharmaceutically-acceptable” refers to molecular entities and compositions that do not produce a significant allergic or similar untoward reaction when administered to a human. Typically, such compositions are prepared as injectables, either as liquid solutions or suspensions; solid forms suitable for solution in, or suspension in, liquid prior to injection can also be prepared. The preparations can also be emulsified.

As used herein, “carrier” includes any and all solvents, dispersion media, vehicles, coatings, diluents, isotonic and absorption delaying agents, buffers, carrier solutions, suspensions, colloids, and the like. Except insofar as any conventional media or agent is incompatible with the active ingredient, its use in the compositions is contemplated.

Methods of Treatment. Certain embodiments include methods of treating a disease or condition in a subject in need thereof, comprising administering a final IV solution to a subject in need thereof via a polyolefin IV bag, as described herein.

In certain embodiments, the subject in need thereof has an ischemic condition, for example, an ischemic condition selected from one or more of brain ischemia (ischemic stroke), cardiac ischemia (myocardial ischemia), ischemic colitis, limb ischemia, and cutaneous ischemia. In particular embodiments, the subject in need thereof has a chronic kidney disease (CKD), acute kidney injury, or a cardio-renal disease. In certain embodiments, the subject in need thereof has a hemorrhagic condition, for instance, hemorrhagic stroke, including intracerebral (within the brain) hemorrhagic stroke and subarachnoid hemorrhagic stroke.

In some embodiments, the subject in need thereof has an inflammatory disease or condition. In particular embodiments, the subject in need thereof has a disease or condition selected from (essential) hypertension, dementia such as vascular dementia, or mild cognitive impairment (MCI).

In some embodiments, the subject in need thereof has a pregnancy disorder (see, for example, U.S. Provisional Application No. 63/626,954, incorporated by reference in its entirety), including a variety of underlying conditions and disorders related to related to hypertension, metabolic disorders, endothelial injury, reduced fetal blood flow, and other pathologies. Examples of pregnancy disorders include fetal growth restriction (FGR), preeclampsia, eclampsia, post-partum preeclampsia, chronic hypertension, chronic hypertension superimposed with preeclampsia, and gestational hypertension, among others.

Certain embodiments include intravenously administering the final IV solution to the subject in need thereof at a total KLK1 polypeptide intravenous dosage of about 0.1 μg/kg to about 0.25 μg/kg to about 0.5 μg/kg to about 5 μg/kg to about 10.0 μg/kg to about 15 μg/kg or to about 20 μg/kg, optionally about 0.1, 0.2, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19 or 20 μg/kg. In certain embodiments, the final IV solution is formulated or administered at a total KLK1 polypeptide intravenous dosage of about 0.25 or 0.5 μg/kg.

In certain instances, IV administration of a composition achieves in the subject a therapeutically-effective serum level of the one or more KLK1 polypeptides. In some instances, IV administration achieves a therapeutically-effective serum level of the one or more KLK1 polypeptides in about or less than about 0.5, 1, 2, 3, 4, 5, 6, 7, 8, 9, 10, 11, 12, 13, 14, 15, 16, 17, 18, 19, or 20 hours following administration. In some instances, the therapeutically-effective serum level is about or at least about 1.0 to about or at least about 5.0 ng/ml, or about or at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mg/ml, including all ranges in between.

Certain embodiments include a dosage regimen of administering one or more compositions at defined intervals over a period of time. For example, certain dosage regimens include intravenously administering once or twice a day, once or twice every two days (e.g., once a day every other day), once or twice every three days (e.g., once a day every third day following an initial or earlier administration), once or twice every four days, once or twice every five days, once or twice every six days, once or twice every week, once or twice every other week. Specific dosage regimens include intravenously administering once a day every three days (e.g., once a day every third day following an initial or earlier administration).

Some variation in dosage will necessarily occur depending on the condition of the subject being treated. The person responsible for administration will, in any event, determine the appropriate dose or dosing regimen for the individual subject. Moreover, for human administration, preparations should meet sterility, pyrogenicity, and general safety and purity standards as required by the FDA. In some instances, preparation are substantially endotoxin-free or pyrogen-free, as described herein. According to the FDA Guidance for Industry; Estimating the Maximum Safe Starting Dose in Initial Clinical Trial for Therapeutics in Adult Healthy Volunteers (July 2005), Appendix D: Converting animal doses to human equivalent doses. A human equivalent dose is 1/7 the rat dose and a human equivalent dose is 1/12 a mouse dose.

In some embodiments, intravenously administering a composition improves one or more clinical parameters in the patient. In certain embodiments, the one or more clinical parameters are selected from decreased albuminuria (UACR), increased estimated glomerular filtration rate (eGFR), decreased blood pressure, serum KLK1 levels of about 1-5 ng/ml, decreased swelling, including in the lower extremities of the patient, and decreased risk or occurrence of cardiovascular events in the patient, including decreased risk or occurrence of myocardial infarction or stroke. In specific embodiments, administering the composition decreases UACR in the subject by about or at least about 25, 30, 35, 40, 45, 50, 55, 60, 65, or 70% or more. Any one or more of the foregoing clinical parameters can be measured according to routine clinical techniques in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Evaluating the Effects of Surfactants on DM199 in Polyolefin IV Bags

Experiments were performed to evaluate the potential of surfactants to reduce the loss of DM199 active in the IV bag admixture due to adsorption by polyolefins. Polysorbate 20 (PS20) or polysorbate 80 (PS80) were added to a 50-mL saline, polyolefin IV bag prior to dosing with DM199 drug product and then the admixture was analyzed for the active drug by SEC-HPLC and particulate matter. Polyolefin IV bags containing the DM199 active at 0.5 μg/mL (50 kg patient dose) or 1.0 μg/mL (100 kg patient dose) were evaluated with surfactants added in the concentration range of 0.003%-0.100% for PS20 and 0.001%-0.010% for PS80. Each admixture was analyzed initially (immediately after preparation) and after being stored in the bag at room temperature under ambient lighting for eight hours.

DM199 was provided at an initial concentration of 0.5 mg/mL. Stock solutions of diluted DM199 were prepared at 0.025 mg/ml and 0.05 mg/mL in saline (scaled as needed) for dosing in 50 mL IV bags. Spiking solutions of PS20 and PS80 were prepared by diluting into water (scaled as needed) for use in adding the surfactants to the IV bags.

The preparation of IV bags were performed in a Biosafety cabinet to reduce the potential for particulate matter contamination. IV bags were prepared at two DM199 concentrations in saline containing either PS20 or PS80 surfactant at three different concentrations. Each 50-mL IV bag was prepared by adding a surfactant (either PS20 or PS80) to the bag at three different concentrations prior to dosing with the DM199 drug product (see Table E1 below). The IV bag were gently inverted multiple times to check for leaks.

TABLE E1
IV Bag Preparations

| | | Final Surfactant |
|---|---|---|
| DM199 concentration | Sample Description | Concentration |
| N/A | Saline | — |
| | PS20 | 0.003% |
| | PS20 | 0.100% |
| | PS80 | 0.001% |
| | PS80 | 0.010% |
| 1.0 μg/mL | Control | — |
| | PS20 | 0.003% |
| | | 0.020% |
| | | 0.100% |
| | PS80 | 0.001% |
| | | 0.003% |
| | | 0.010% |
| 0.5 μg/mL | Control | — |
| | PS20 | 0.003% |
| | | 0.020% |
| | | 0.100% |
| | PS80 | 0.001% |
| | | 0.003% |
| | | 0.010% |

After dosing the saline IV bags containing surfactant, the admixture was analyzed immediately (initial T=0 time point) and after being stored under room temperature/ambient lighting conditions for eight hours (T=8).

Particulate Matter (HIAC). Particulate matter testing was performed per cUSP <787>, Subvisible Particulate Matter in Therapeutic Protein Injections, following Method 1 (light obscuration). The results are shown in Table E2 below.

TABLE E2
Particulate Matter

| | | | Particulate Matter (No.) | | | |
|---|---|---|---|---|---|---|
| | | | | | 8 Hours | |
| DM199 | Sample | Surfactant | Initial | | | ≥25 |
| concentration | Description | Concentration | ≥10 nm | ≥25 nm | ≥10 nm | nm |
| Control | Saline | — | 67 | 0 | 184 | 0 |
| | PS20 | 0.003% | 100 | 17 | 17 | 0 |
| | | 0.100% | 184 | 17 | 117 | 0 |
| | PS80 | 0.001% | 100 | 17 | 17 | 0 |
| | | 0.010% | 117 | 0 | 100 | 0 |
| 1.0 μg/mL | Control | — | 0 | 0 | 183 | 17 |
| | PS20 | 0.003% | 0 | 0 | 67 | 50 |
| | | 0.020% | 284 | 34 | 500 | 50 |
| | | 0.100% | 50 | 0 | 83 | 17 |
| | PS80 | 0.001% | 0 | 0 | 100 | 0 |
| | | 0.003% | 17 | 0 | 33 | 0 |
| | | 0.010% | 0 | 0 | 100 | 0 |
| 0.5 μg/mL | Control | — | 33 | 0 | 83 | 0 |
| | PS20 | 0.003% | 33 | 0 | 100 | 0 |
| | | 0.020% | 217 | 0 | 83 | 17 |
| | | 0.100% | 17 | 0 | 133 | 0 |
| | PS80 | 0.001% | 0 | 0 | 17 | 0 |
| | | 0.003% | 17 | 0 | 117 | 0 |
| | | 0.010% | 33 | 0 | 633 | 17 |

SEC-HPLC Assay. Size Exclusion HPLC assay was performed in duplicate by HPLC analysis per Alcami TP81234, Assay Determination of DM199 Drug Product Admixture (0.2-4.0 μg/mL in 0.9% NaCl IV Bags). The results are shown in Table E3 below.

TABLE E3
SEC-HPLC Assay Results

| | | | DM199 (μg/mL) | | | | | |
|---|---|---|---|---|---|---|---|---|
| DM199 | | | 0 Hours | | | 8 Hours | | |
| concentration | Sample | Surfactant | Prep | Prep | Mean | Prep | Prep | Mean |
| (dosing level) | Description | Concentration | 1 | 2 | (2) | 1 | 2 | (2) |
| Control | Saline | — | 0.00 | 0.00 | 0.00 | 0.00 | 0.00 | 0.00 |
| | PS20 | 0.003% | 0.00 | 0.00 | 0.00 | 0.00 | 0.00 | 0.00 |
| | | 0.100% | 0.00 | 0.00 | 0.00 | 0.00 | 0.00 | 0.00 |
| | PS80 | 0.001% | 0.00 | 0.00 | 0.00 | 0.00 | 0.00 | 0.00 |
| | | 0.010% | 0.00 | 0.00 | 0.00 | 0.00 | 0.00 | 0.00 |
| 1.0 μg/mL | Control | — | 0.49 | 0.49 | 0.49 | 0.52 | 0.52 | 0.52 |
| | PS20 | 0.003% | 1.12 | 1.11 | 1.11 | 1.10 | 1.10 | 1.10 |
| | | 0.020% | 1.26 | 1.22 | 1.24 | 1.24 | 1.21 | 1.22 |
| | | 0.100% | 1.26 | 1.20 | 1.23 | 1.30 | 1.20 | 1.25 |
| | PS80 | 0.001% | 1.03 | 1.02 | 1.03 | 1.05 | 1.02 | 1.04 |
| | | 0.003% | 1.09 | 1.09 | 1.09 | 1.10 | 1.10 | 1.10 |
| | | 0.010% | 1.06 | 1.13 | 1.09 | 1.16 | 1.18 | 1.17 |
| 0.5 μg/mL | Control | — | 0.21 | 0.20 | 0.20 | 0.15 | 0.15 | 0.15 |
| | PS20 | 0.003% | 0.52 | 0.52 | 0.52 | 0.56 | 0.55 | 0.55 |
| | | 0.020% | 0.64 | 0.70 | 0.67 | 0.64 | 0.68 | 0.66 |
| | | 0.100% | 0.76 | 0.72 | 0.74 | 0.76 | 0.72 | 0.74 |
| | PS80 | 0.001% | 0.47 | 0.45 | 0.46 | 0.53 | 0.51 | 0.52 |
| | | 0.003% | 0.51 | 0.48 | 0.50 | 0.54 | 0.52 | 0.53 |
| | | 0.010% | 0.51 | 0.48 | 0.50 | 0.54 | 0.57 | 0.56 |

Similar studies were performed with lower concentrations of PS80 and also to evaluate the effects in glass (volumetric flask) as a substrate. Table E4 below shows the results calculated against standard.

TABLE E4
SEC-HPLC Assay Results

| DM199 | | | | | |
|---|---|---|---|---|---|
| concentration | Sample | Surfactant | DM199 (μg/mL) | | |
| (dosing level) | Description | Concentration | Prep 1 | Prep 2 | Mean (2) |
| 0.5 μg/mL | Control | — | 0.43 | 0.41 | 0.42 |
| (Glass Flask) | PS80 | 0.0005% | 0.69 | 0.62 | 0.66 |
| | | 0.0015% | 0.60 | 0.50 | 0.55 |
| | | 0.00025% | 0.57 | 0.60 | 0.58 |
| | | 0.00075% | 0.63 | 0.64 | 0.63 |
| Control | Saline | — | 0 | 0 | 0 |
| (50-mL IV bag) | PS80 | 0.0005% | 0 | 0 | 0 |
| | | 0.0015% | 0 | 0 | 0 |
| 0.5 μg/mL | Control | — | 0.30 | 0.29 | 0.30 |
| (50-mL IV bag) | PS80 | 0.0005% | 0.66 | 0.68 | 0.67 |
| | | 0.0015% | 0.73 | 0.75 | 0.74 |
| 0.5 μg/mL | Control | — | 0.25 | 0.22 | 0.24 |
| (100-mL IV bag) | PS80 | 0.00025% | 0.64 | 0.66 | 0.65 |
| | | 0.00075% | 0.73 | 0.75 | 0.74 |

Results. IV bags or glass contains without being treated with either surfactant have significantly lower recovery of DM199 than the IV bags or glass containers treated with surfactant, as low as 20% of expected bag concentration. The lower percent recovery is particularly evident at the lower dose concentrations. The eight hour incubation time does not appear to significantly impact the recovery of DM199. The lowest concentration of each surfactant has been shown to significantly increase the recovery of DM199 with minimal interference with the active peak for quantitation for both 0.5 μg/mL and 1.0 μg/mL dosing levels

1. A pharmaceutical composition, comprising:
(i) one or more tissue kallikrein (KLK1) polypeptides; and
(ii) a polysorbate surfactant (PS) optionally selected from PS20 and PS80.

2-8. (canceled)

9. The pharmaceutical composition of claim 1, wherein
(i) is at concentration of about 100 μg/mL, (ii) is at a concentration of about 0.43% to about 0.87% (for use with 50 mL IV bag), and the composition is in a vial at a total volume of about 0.15 mL to about 0.5 mL; or
(i) is at concentration of about 100 μg/mL, (ii) is at a concentration of about 0.86% to about 1.74% (for use with 100 mL IV bag), and the composition is in a vial at a total volume of about 0.15 mL to about 0.5 mL.

10. The pharmaceutical composition of claim 1, wherein (i) is DM199.

11-12. (canceled)

13. A method of preparing a polyolefin intravenous (IV) bag for intravenous administration to a subject in need thereof, wherein the polyolefin IV bag comprises an IV solution, comprising:
(a) adding the pharmaceutical composition of claim 1 to the IV solution in the polyolefin IV bag; and
(b) inverting or agitating the polyolefin IV bag for a time sufficient to disperse the pharmaceutical composition into a final IV solution.

14-19. (canceled)

20. The method of claim 13, wherein
the pharmaceutical composition of step (a) comprises the one or more KLK1 polypeptides of (i), optionally the DM199, at concentration of about 100 μg/mL, and the PS surfactant of (ii) at a concentration of about 0.43% to about 0.87%, and wherein the polyolefin IV bag with the final IV solution of step (b) has a total volume of about 50 mL, the one or more KLK1 polypeptides, optionally DM199, are in the final IV solution at a concentration of about 0.15 μg/mL to about 1.0 μg/mL, and the PS surfactant of (ii) is in the final IV solution at a concentration of about 0.001% to about 0.002%; or
the pharmaceutical composition of step (a) comprises the one or more KLK1 polypeptides of (i), optionally DM199, at concentration of about 100 μg/mL, and the PS surfactant of (ii) at a concentration of about 0.86% to about 1.74%, and wherein the polyolefin IV bag with the final IV solution of step (b) has a total volume of about 100 mL, wherein the one or more KLK1 polypeptides of (i), optionally the DM199, are in the final IV solution at a concentration of about 0.15 μg/mL to about 1.0 μg/mL, and the PS surfactant is in the final IV solution at a concentration of about 0.001% to about 0.002%.

21. The method of claim 13, comprising (c) intravenously administering the final IV solution to the subject in need thereof via the polyolefin IV bag, optionally at a total KLK1 polypeptide intravenous dosage of about 0.1 μg/kg to about 0.25 μg/kg to about 0.5 μg/kg to about 5 μg/kg or to about 10.0 μg/kg.

22. A method of treating a disease or condition in a subject in need thereof, comprising administering a final IV solution to the subject in need thereof via the polyolefin IV bag as prepared according to claim 1, optionally at a total KLK1 polypeptide intravenous dosage of about 0.1 μg/kg to about 0.25 μg/kg to about 0.5 μg/kg to about 5 μg/kg or to about 10.0 μg/kg.

23. A patient care kit, comprising:
(i) a first vial comprising a pharmaceutical composition, wherein the pharmaceutical composition comprises one or more tissue kallikrein (KLK1) polypeptides; and
(ii) a second vial comprising a polysorbate surfactant optionally selected from PS20 and PS80.

24-30. (canceled)

31. The patient care kit of claim 23, wherein
the first vial is at a total volume of about 0.15 mL to about 0.5 mL and comprises the pharmaceutical composition of one or more KLK1 polypeptides, optionally DM199, at a concentration of about 100 μg/mL, and
the second vial is at a total volume of about 0.25 mL to about 2.0 mL and comprises the polysorbate surfactant at a concentration of about 0.1% to about 0.8%, optionally wherein the second vial is about 2 mL total volume/about 0.1% surfactant, about 1 mL total volume/about 0.2% surfactant, about 0.5 mL total volume/about 0.4% surfactant, or about 0.25 mL total volume/about 0.8% surfactant.

32. The patient care kit of claim 23, wherein the pharmaceutical composition of (i) comprises DM199.

33-34. (canceled)

35. A method of preparing a polyolefin intravenous (IV) bag for intravenous administration to a subject in need thereof, wherein the polyolefin IV bag comprises an IV solution, comprising:
(a) adding a polysorbate surfactant to the IV solution in the IV bag, wherein the polysorbate surfactant is selected from PS20 and PS80;
(b) separately adding a pharmaceutical composition comprising one or more tissue kallikrein (KLK1) polypeptides to the IV solution in the polyolefin IV bag; and
(c) inverting or agitating the IV bag for a time sufficient to disperse the pharmaceutical composition and the polysorbate surfactant into a final IV solution.

36-41. (canceled)

42. The method of claim 35, wherein the polyolefin IV bag with the final IV solution of (c) has a total volume about 50 ml or 100 mL, the polysorbate surfactant added from (a) is in the final IV solution at a concentration of about 0.001% to about 0.002%, and the one or more KLK1 polypeptides added from (b), optionally DM199, are in the final IV solution at a concentration of about 0.15 μg/mL to about 1.0 μg/mL.

43-44. (canceled)

45. A method of treating a disease or condition in a subject in need thereof, comprising administering a final IV solution to the subject in need thereof via the polyolefin IV bag as prepared according to claim 35, optionally at a total KLK1 polypeptide intravenous dosage of about 0.1 μg/kg to about 0.25 μg/kg to about 0.5 μg/kg to about 5 μg/kg or to about 10.0 μg/kg.

46. A polyolefin intravenous (IV) bag, comprising a final IV solution of:
(i) one or more tissue kallikrein (KLK1) polypeptides; and
(ii) a polysorbate surfactant selected from PS20 and PS80.

47-52. (canceled)

53. The polyolefin IV bag of claim 46, wherein the final IV solution has a total volume about 50 ml or 100 mL, the one or more KLK1 polypeptides from (i), optionally DM199, are in the final IV solution at a concentration of about 0.15 μg/mL to about 1.0 μg/mL, and the polysorbate surfactant from (ii) is in the final IV solution at a concentration of about 0.001% to about 0.002%.

54. The polyolefin IV bag of claim 46, wherein (i) is DM199.

55-56. (canceled)

57. A method of treating a disease or condition in a subject in need thereof, comprising administering a final IV solution to the subject in need thereof via the polyolefin IV bag of claim 46, optionally at a total KLK1 polypeptide intravenous dosage of about 0.1 μg/kg to about 0.25 μg/kg to about 0.5 μg/kg to about 5 μg/kg or to about 10.0 μg/kg.

58. The pharmaceutical composition of claim 1, wherein the one or more KLK1 polypeptides comprise a first KLK1 polypeptide and a second KLK1 polypeptide, wherein the first KLK1 polypeptide has three N-linked glycans attached at residues 78, 84, and 141 and the second KLK1 polypeptide has two N-linked glycans attached at residues 78 and 84 but not 141, and wherein the first KLK1 polypeptide and the second KLK1 polypeptides are present at a ratio of 45:55 to 55:45.

59-62. (canceled)

63. The pharmaceutical composition of claim 58, wherein the first KLK1 polypeptide and the second KLK1 polypeptides are present at a ratio of about 50:50.

64-68. (canceled)

69. The pharmaceutical composition of claim 58, wherein the KLK1 polypeptide(s) comprise, consist, or consist essentially of SEQ ID NO: 3 or 4, or an active fragment or variant thereof having at least 95% sequence to SEQ ID NO: 3 or 4.

70. (canceled)

71. The method of claim 13, wherein the subject in need thereof has an ischemic condition, optionally selected from one or more of brain ischemia (ischemic stroke), cardiac ischemia (myocardial ischemia), ischemic colitis, limb ischemia, and cutaneous ischemia.

72. The method of claim 13, wherein the subject in need thereof has a chronic kidney disease (CKD), acute kidney injury, or a cardio-renal disease.

73. The method of claim 13, wherein the subject in need thereof has a hemorrhagic condition, optionally hemorrhagic stroke, including intracerebral (within the brain) hemorrhagic stroke and subarachnoid hemorrhagic stroke.

74. The method of claim 13, wherein the subject in need thereof has an inflammatory disease or condition.

75. The method of claim 13, wherein the subject in need thereof has a disease or condition selected from (essential) hypertension, dementia optionally vascular dementia, and mild cognitive impairment (MCI).

76. The method of claim 13, wherein the subject in need thereof has a pregnancy disorder, optionally fetal growth restriction (FGR), preeclampsia, eclampsia, post-partum preeclampsia, chronic hypertension, chronic hypertension superimposed with preeclampsia, and gestational hypertension.
